(19) United States
(12) Patent Application Publication
(10) Pub. No.: US 2026/0295018 A1
(43) Pub. Date: Oct. 1, 2026

(54) EXTRACELLULAR VESICLES COMPRISING 15-LIPOXYGENASE

(71) Applicant: ASTON UNIVERSITY, Birmingham, West Midlands (GB)

(72) Inventors: Andrew DEVITT, Dorridge, Solihull, West Midlands (GB); Ivana MILIC, Birmingham, West Midlands (GB)

(21) Appl. No.: 19/478,625

(22) PCT Filed: Apr. 24, 2024
(86) PCT No.: PCT/GB2024/051055

§ 371 Date: Oct. 24, 2025

(30) Foreign Application Priority Data
Apr. 25, 2023 (GB) 2306026.2

(51) Int. Cl. A61K 38/44; A61K 35/28; A61P 11/00; A61P 37/00

(52) U.S. Cl. CPC A61K 38/44; A61K 35/28; A61P 11/00; A61P 37/00

(57) ABSTRACT

The present invention provides a method of treating an inflammatory condition or disease comprising administering a pharmaceutically effective amount of an extracellular vesicle (EV), the EV comprising 15-lipoxy genase (15-LOX) and/or a nucleic acid encoding 15-LOX, and optionally additionally comprising one or more of 12-lipoxygenase (12-LOX) and 5-lipoxygenase (5-LOX), and/or one or more nucleic acids encoding one or more of 12-LOX and 5-LOX. An extracellular vesicle (EV), the EV comprising one or more of 15-lipoxygenase (15-LOX), and/or a nucleic acid encoding 15-LOX, and optionally additionally comprising one or more of 12-lipoxygenase (12-LOX) and 5-lipoxygenase (5-LOX), and/or one or more nucleic acids encoding one or more of 15-LOX, 12-LOX and 5-LOX, for use in treating an inflammatory disease or condition is also provided, as are isolated extracellular vesicles comprising 15-lipoxy genase (15-LOX) and/or a nucleic acid encoding 15-LOX, and optionally additionally comprising one or more of 12-lipoxygenase (12-LOX), 5 lipoxygenase (5-LOX), and/or one or more nucleic acids encoding one or more of 15-LOX, 12-LOX and 5-LOX.

The invention relates to extracellular vesicles (EV) comprising lipoxygenases (LOX), and in particular 15-LOX and optionally 12-LOX and/or 5-LOX, or comprising nucleic acid sequences encoding such LOXs, and their use to treat inflammatory conditions. The EV may be artificial or derived from for example naturally occurring cells such as viable cells or apoptotic cells or mesenchymal stem cells.

Abbreviations used herein: ACdEV—apoptotic cell-derived extracellular vesicle; COX—cyclooxygenase; EV—extracellular vesicles; LM—lipid mediators of inflammation; LOX—lipoxygenase; MΦ—macrophage; LT—leukotriene; LX—lipoxin; PD—protectin; PG—prostaglandin; PS—phosphatidylserine; PUFA-poly—unsaturated fatty acid; SPM—specialised pro-resolving lipid mediators; RV—resolvin; VD₃—1α,25-dihydroxyvitamin D₃.

Programmed cell death, also known as apoptosis, is a physiological process by which infected, damaged or simply unwanted cells are removed. For a long time it was considered a “silent” process, described mainly because of its distinct morphological features¹. Today, it is widely recognised that apoptosis plays a central role in the homeostatic regulation of immune responses². Hence, failure to execute apoptosis appropriately risks significant consequences such as chronic inflammation, autoimmunity³ and tumourigenesis⁴.

Inflammation is a complex and carefully orchestrated process at both cellular and molecular levels, and it is a protective immune response to an acute challenge which, if is not controlled efficiently, becomes chronic, leading to inflammatory disease. Release of exogenous or endogenous chemical mediators within injured tissues promotes the inflammatory response through local vascular changes to enable recruitment of professional phagocytes (i.e. neutrophils and macrophages). However, once they have met and eliminated an immune challenge, the tissue must return to its pre-inflamed state through the resolution phase of inflammation. For inflammation to resolve, continued inflammatory cell recruitment is halted, recruited neutrophils undergo apoptosis and are phagocytosed by resident and recruited macrophages which exit the tissue via the lymphatics. Thus apoptosis has a central role in re-establishing tissue homeostasis by interacting with macrophages and promoting the resolution stage of inflammation, although the full molecular mechanisms remain to be determined⁵.

The molecular signals that orchestrate the movement of neutrophils and macrophages to the inflamed tissues, include bioactive metabolites known as lipid mediators of inflammation (LM). They are the product of phospholipid catabolismand are released locally by the recruited cells, while some of them can be of microbial origin. The entire resolution process is driven by an overall balance of pro-inflammatory and pro-resolving lipid mediators of inflammation secreted as a consequence of the coordinated actions of cell receptors and enzymes⁶. During the acute phase of inflammation phagocyte recruitment is supported by the local release of leukotrienes (LTs)⁷ and prostaglandins (PGs)⁸. Their increasing levels help drive inflammation, thus both metabolite classes are known for their pro-inflammatory properties. However, PGD₂ and PGE₂ produced via the cyclooxygenase (COX) pathway can act as a pro-resolving molecular ‘switch’ having the ability to induce the transcription of lipoxygenases (LOX), enzymes responsible for the synthesis of small yet very potent specialist pro-resolving lipid mediators (SPM), such as lipoxins (LXs), resolvins (RVs) and protectins (PDs)⁹. For example, such a “class switch” in neutrophils and macrophages would require upregulation of 15-LOX with a switch from using arachidonic acid (AA) for LTB₄ production (via 5-LOX) towards synthesis of LXA₄ via 15-LOX, which would actively promote further neutrophil uptake, stimulate non-phlogistic monocyte recruitment, support macrophage uptake of apoptotic neutrophils and facilitate their exit via lymphatics. Neutrophil apoptosis and its communication with the innate immune system therefore represents a keystone event that sets the scene for guiding inflammation to a successful conclusion resolution¹⁰.

The nature of the complex intercellular communication between dying cells and the immune system remains to be defined. Beside lipid mediators, dying cells drive phagocyte recruitment by releasing so called soluble “find-me” signals (ATP and UTP, fractalkine, lysophosphatidylcholine, sphingosine 1-phosphate), and display “eat-me” tags on their surface to facilitate phagocytic uptake¹¹. In recent years, extracellular vesicles have emerged as novel mediators of cellular communication¹². Given the nature of their interaction with a host ‘recipient’ cell, extracellular vesicles may not only trigger surface receptors on immune cells, they may also deliver their myriad components and cargo of proteins, lipids, small molecules and genetic material, thereby inducing immune responses¹³. Whilst extracellular vesicles from viable cells have been studied in more detail, apoptotic cell-derived extracellular vesicles (ACdEV) and their immunomodulatory properties have received relatively little attention¹⁴. Up to now, different research groups have demonstrated chemoattractive properties of ACdEV^{15,16,17}. Fractalkine (CX3CL1)^{16,18} and ICAM-3¹⁷ on the surface of ACdEV have been identified as “find-me” signals responsible for macrophage migration and ACdEV association with immune cells, while glycosylated ligands on the ACdEV surface acted as “eat-me” signals¹⁹. It is quite plausible, that a range of other ACdEV-associated molecules could have immunomodulatory properties.

The inventors investigated release of EV from dying leukocytes and sought to define their composition with a focus on their ability to communicate with the immune system and actively promote resolution of inflammation. The inventors show that during apoptosis human primary T cells actively release ACdEV with a “pro-resolving phenotype” characterised by a presence of active lipoxygenases while carrying an overall lipid metabolome with higher levels of SPM compared to the pro-inflammatory LTs and PG. They also show carriage of LOX in EV from a range of leukocytes including monocytes and B cells primary origin and cell lines. These “active EV” carry active lipoxygenases, with highest specific activity of 15-LOX, and deliver active lipoxygenases to recipient cells to support AA catabolismimportantly, they reveal that these ACdEV interact with macrophages thereby significantly reducing their endogenous levels of LTs while driving a pro-resolving phenotype. Taken together these data reveal key novel metabolic activity of ACdEV.

The identification of the key elements required to produce pro-resolving activity allows the production of EV having the ability to be used to reduce inflammatory conditions.

The invention provides a method of treating an inflammatory disease or condition comprising administering a pharmaceutically effective amount of an extracellular vesicles (EV), the EV comprising one or more of 15-lipoxygenase (15-LOX), 12-lipoxygenase (12-LOX) and/or 5-lipoxygenase (5-LOX) and/or one or more nucleic acid encoding one or more of 15-LOX, 12-LOX and/or 5-LOX. Preferably, the EV comprises 15-lipoxygenase (15-LOX) and/or a nucleic acid encoding 15-LOX, and optionally additionally comprises one or more of 12-lipoxygenase (12-LOX) and 5-lipoxygenase (5-LOX), and/or one or more nucleic acids encoding one or more of 12-LOX and 5-LOX. 15-LOX (encoded by ALOX15 or arachidonate 15-lipoxygenase) catalyses the oxygenation of arachidonic at the carbon 15 to form 15-HpETE. 15-LOX may be used alone.

15-LOX may also have the ability to catalyse the oxygenation of arachidonic acid at the carbon 12. Alternatively, or additionally, 15-LOX may be used with arachidonate 12-lipoxygenase (12-LOX) having the ability to oxidise arachidonic acid at position 12.

Arachidonate 5-lipoxygenase, also known as ALOX-5, 5-lipoxygenase or 5-LOX is known to transform a number of essential fatty acids into leukotrienes as well as a wide range of other biologically active components. It possesses two catalytic activities. On arachidonic acid, it adds a hydroperoxyl to carbon 5 of arachidonic acid. The 5 S-HpETE intermediate may then be released by the enzyme and rapidly reduced by cellular glutathione peroxidases to its corresponding alcohol, or alternatively, further metabolised by the enzymes epoxidase activity to its epoxide. The enzyme is thought to have both pro-inflammatory and resolving activity (that is, reduces inflammation). Accordingly, the presence of 5-LOX may be beneficial in promoting pro-resolution phenotypes and reducing inflammation. One possibility is to inhibit, or provide, an inhibitor of, 5-LOX to reduce the pro-inflammatory activity of 5-LOX. The lipoxygenase typically has the ability to oxidise both free arachidonic acid (AA) as well as AA bound to phospholipids and lipoproteins. The nucleic acids may encode one or more of 15-LOX or 12-LOX or 5-LOX. The or each nucleic acid typically encodes an active lipoxygenase. One or both of 15-LOX and 12-LOX may be typically used. Preferably 15-LOX is used, optionally in combination with one or more of 12-LOX and 5-LOX.

The nucleic acid may be a ribonucleic acid (RNA). Optionally the RNA may be messenger RNA (mRNA).

Extracellular vesicles comprising one or more of 5-lipoxygenase, 12-lipoxygenase and 15-lipoxygenase, or one or more nucleic acid encoding one or more of 15-LOX, 12-LOX and 5-LOX for use to treat an inflammatory disease or condition are also provided. Preferably, the EV comprises 15-lipoxygenase (15-LOX) and/or a nucleic acid encoding 15-LOX, and optionally additionally comprises one or more of 12-lipoxygenase (12-LOX) and 5-lipoxygenase (5-LOX), and/or one or more nucleic acids encoding one or more of 12-LOX and 5-LOX.

The inflammatory disease may be a chronic inflammatory disease or condition. The inflammatory disease may be selected from a chronic wound, a cutaneous inflammatory disease, an autoimmune disease, nephritis, ageing and dementia. It is noted that many signs of ageing involve inflammatory processes. The ability to use either 15-LOX, 12-LOX and 5-LOX and/or any of their combinations to promote co-resolving phenotypes to reduce inflammation is expected to be able to treat one or more ageing-related conditions. The inflammatory disease may be selected from an internal or external wound, a diabetic wound, and SLE (Systemic Lupus Erythematosus).

The EV may comprise 15-lipoxygenase (15-lox) or a nucleic acid encoding 15-lox.

The EV may be an isolated, naturally occurring, EV or a non-naturally occurring EV. For example, the EV may be a cell-derived EV, such as an apoptotic cell-derived extracellular vesicle (ACdEV), or a mesenchymal stem cell EV. Both types of EV are generally known in the art.

### The EV may be an artificial, non-naturally occurring EV.

Extracellular vesicles are lipid bilayer-delimited particles that are naturally released from almost all types of cell, but unlike a cell cannot replicate. Naturally occurring EVs typically carry a cargo of proteins, nucleic acids, lipids, metabolites, or other components from a parent cell. It is also possible to produce artificial EV's comprising a lipid bilayer and one or more additional components.

For example, EVOX Therapeutics, have a number of proprietary EV systems for transporting mRNA or proteins, such as active enzymes. See, for example, WO 2020/225392 and WO 2022/229220, and WO 2018/153581, incorporated herein in their entirety.

Accordingly, the EV may be sourced from naturally or non-naturally occurring sources. The EV may be targeted to be absorbed by pro-inflammatory macrophages. Accordingly, they may comprise one or more markers capable of targeting pro-inflammatory macrophages. These may be one or more “eat me” markers, such as Annexin A1, calrecticulin and phosphatidylserine. One or more adhesion molecules may be provided on the EV, such as an IgSF member, most typically ICAM-3 (intercellular adhesion molecule 3), CD44, intergrin α-1 and integrin β-2.

One or more “find me” molecules may also be present on the EV surface, such as Fractalkine (CX3CL1) and ICAM-3.

It is desirable to exclude pro-inflammatory compounds. These include, for example, leukotrienes, such as arachidonate 5-lipoxygenase activating protein (AL5AP, also known as FLAP) and leukotriene A-4 hydrolase (LKHA4). Flap may be present when 5-LOX is used in the EV as it has a role in 5-LOX activity.

The EV typically has a size range of 10-1,000 nm, more typically 70-700 nm.

Typically, EV produced from naturally occurring sources (such as ACdEV or mesenchymal stem cells EV) have a diameter in the range of about 90 nm to about 130 nm, more typically about 100 nm to about 200 nm. Optionally, EV produced from naturally occurring sources (such as ACdEV or mesenchymal stem cells EV) have a mode size in the range of 90 nm to 130 nm, more typically 100 nm to 200 nm.

The EV may be administered to a subject, or be capable of being administered to a subject intravenously, intraperitonially, topically, intranasally, via oral inhalation, and may be administered by a nebuliser or via an inhaler.

The invention also provides isolated EV comprising one or both of 15-LOX or 12-LOX, or one or more nucleic acids comprising one or both of 15-LOX or 12-LOX. Preferably the isolated EV comprise 15-LOX, optionally in combination with 12-LOX.

They may also comprise 5-LOX, as discussed above. Moreover, as discussed previously, the EV may be an isolated naturally occurring EV or non-naturally occurring EV as defined above.

The EV may be provided with a pharmaceutically acceptable excipient.

A naturally occurring EV may be modified to remove one or more unwanted, or undesirable, pro-inflammatory molecules, such as those defined above or incorporate inhibitors of such pro-inflammatory molecules.

The EV may consist essentially of 15-LOX and/or 12-LOX, optionally with or without 5-LOX. Optionally the EV may consist essentially of 15-LOX. The EV may consist essentially of 15-LOX in combination with one or more of 12-LOX and 5-LOX.

The EV may optionally be a naturally occurring EV, such as such as ACdEV or mesenchymal stem cells (MSC) EV. Without being bound by theory, the present inventors believe that EV derived from apoptotic cells may be capable of other uptake mechanisms by virtue of their membrane lipid organisation. MSC-derived EV may also exert different effects through carriage of other immunomodulatory molecules e.g. INDO.

One or more markers capable of targeting pro-inflammatory macrophages may be provided, such as one or more “eat-me” markers, such as annexin A1, calreticulin or phosphatidylserine.

One or more “find me” molecules may also be present on the EV surface, such as Fractalkine (CX3CL1) and ICAM-3.

Typically, the EV has a diameter of about 10 nm to about 1,000 nm, more typically about 70 nm to about 700 nm. The EV may have a diameter of about 90 nm to about 130 nm, more typically about 100 nm to about 200 nm. Optionally, the EV may have a mode size of 10 nm to 1,000 nm, or 70 nm to 700 nm. The EV may have a mode size of 90 nm to 130 nm, or 100 nm to 200 nm.

The EV may be capable of being administered to a subject interperitoneally, topically intranasally by oral inhalation, and more typically via a nebuliser or an inhaler.

The inventor's in vivo studies show beneficial effects with only 10 μg of EV (as defined by protein concentration) dose per animal (equating to circa 10e⁹ EV from circa 50 million cells). Typically, up to 100 μg is used in the field, 30-50 more typically.

The invention will now be described by way of example with reference to the following figures:

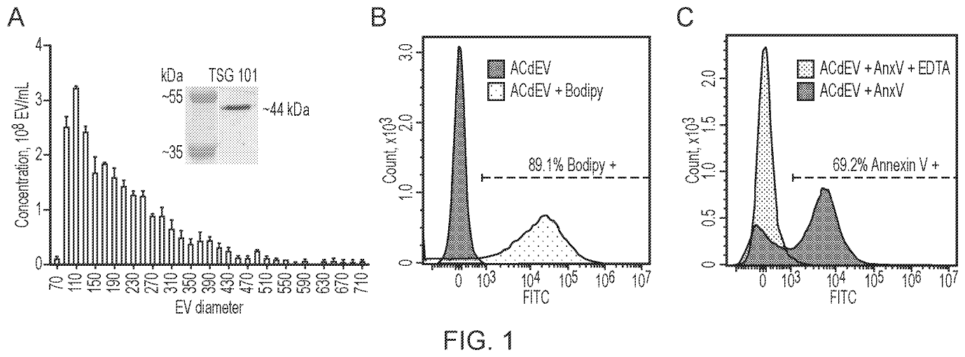

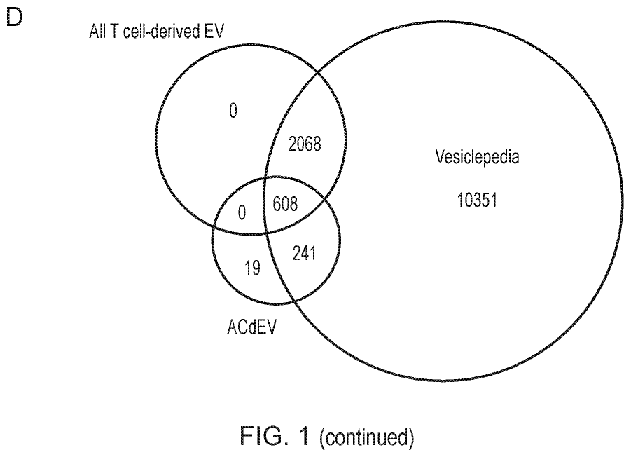

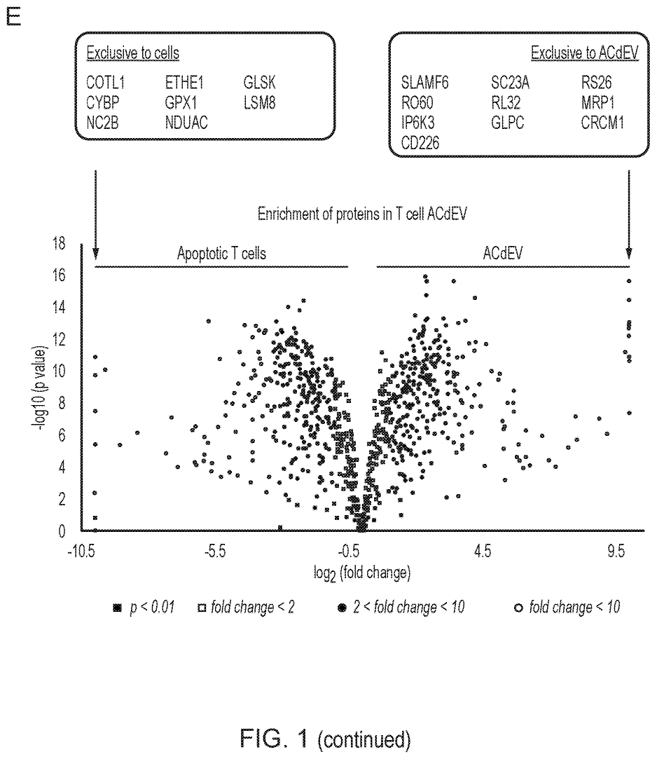

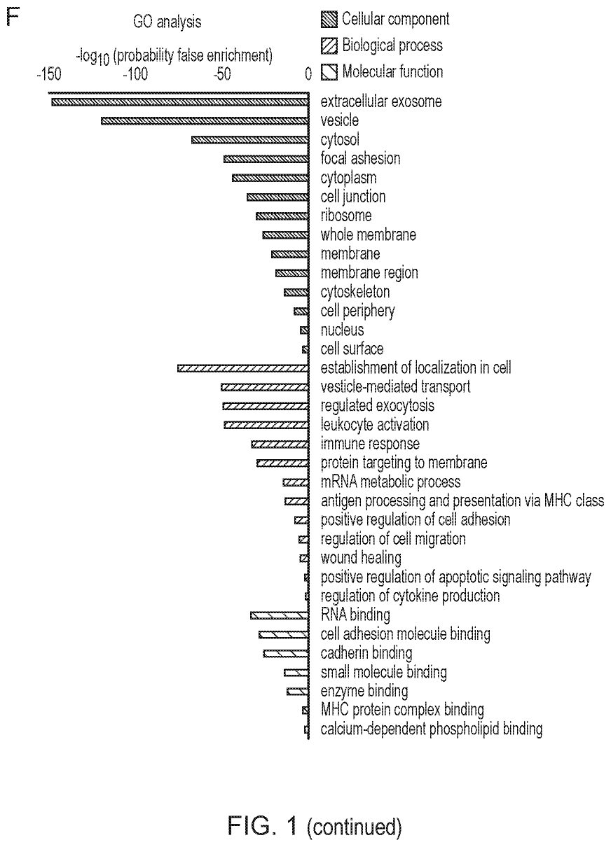

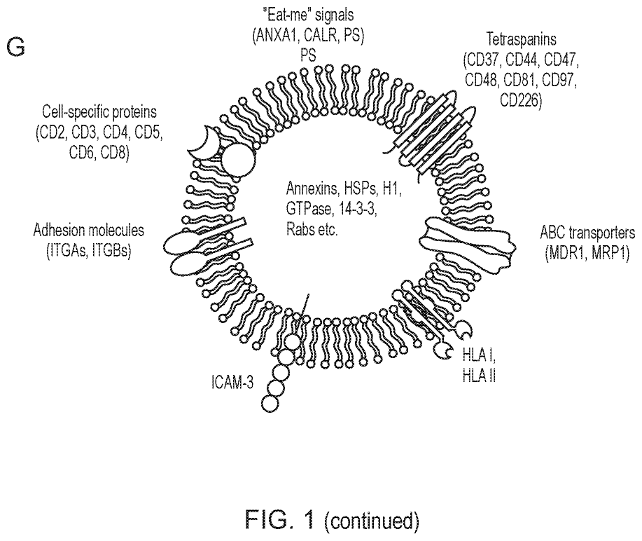

FIG. 1. Characterisation of EV derived from T cells during apoptosis

Primary human T cells were induced to apoptosis with anti-Fas in the presence of cycloheximide. AcdEV were isolated and analysed for size, concentration, PS exposure and protein composition. (A) TRPS measurement of size and concentration of EV isolated by size exclusion chromatography from apoptotic T cell supernatants. Inset: Western blot for TSG-101 indicates that a portion of isolated EV is of exosomal origin. (B) Representative flow cytometry histogram of harvested EV stained with thiol-reactive dye Bodipy indicates that around 89% of measured particles were positively stained and of vesicular origin. Unstained AcdEV were used as a negative control. (C) Representative flow cytometry histogram of EV stained with annexin V (AnxV) shows 69% of EV have exposed PS. EDTA-depletion of Ca²⁺ to abolish PS recognition by AnxV was used as the negative control. (D) Venn diagrams visualising proteome overlaps between AcdEV, whole Vesiclepedia and all T cell-derived EV. (E) Volcano plot showing significant enrichment of proteins in ACdEV (right side) compared to apoptotic T cells. (F) Gene ontology enrichment of Biological processes (green), cellular components (purple) and molecular function of proteins (orange) that were found to me more than 2-fold higher in ACdEV compared to T cells (simplified graph). (G) Schematic representation of ACdEV with major protein classes enriched in ACdEV in this study. Data shown are representative of similar experiments.

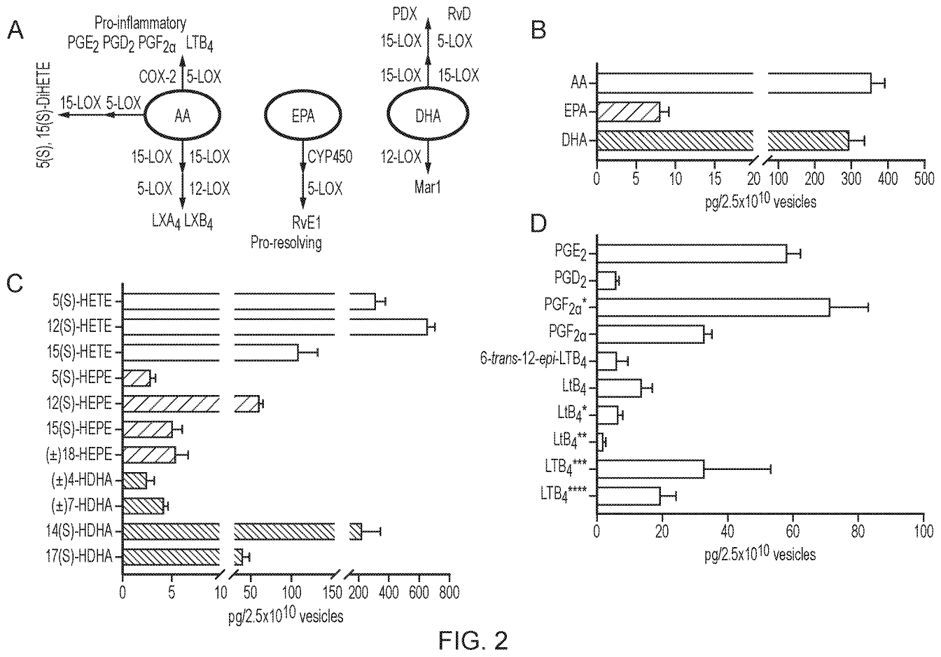

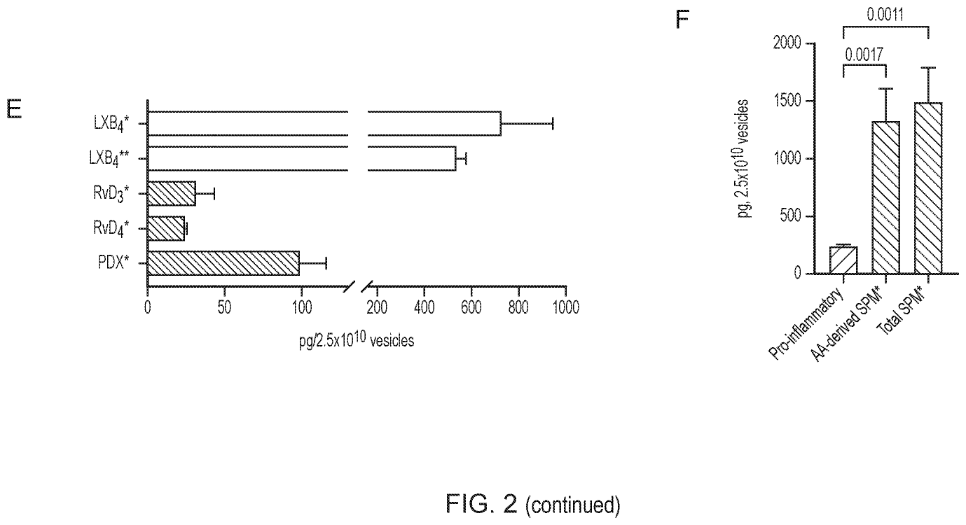

FIG. 2. Lipid metabolome profiling of EV derived from apoptotic primary human T cell

(A) Dual acting SPM (green) and pro inflammatory LM (red) are endogenously produced via LOX and COX pathways from arachidonic (AA), eicosapentaenoic (EPA) and docosahexaenoic (DHA) acids. Targeted LC-MS/MS(MRM) analysis reveals that EV are loaded with PUFA (AA, EPA and DHA); n=4 (B); LOX-oxidized PUFA as primary LOX metabolites (C), pro-inflammatory metabolites (D) and small pro-resolving lipid mediators of inflammation, SI (E); n=5. ACdEV carry higher levels of both AA-derived and total SPM compared to pro-inflammatory metabolites (F). All results are expressed as mean±SEM. Statistics: t-test, two tailed, unpaired.

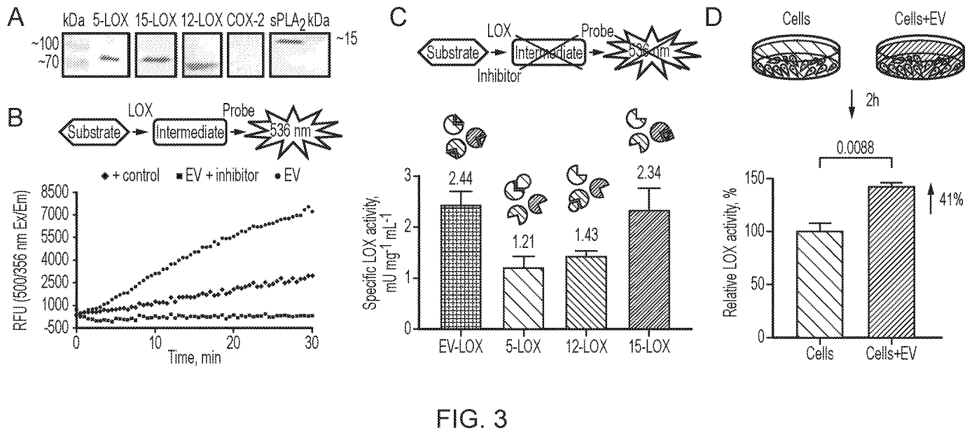

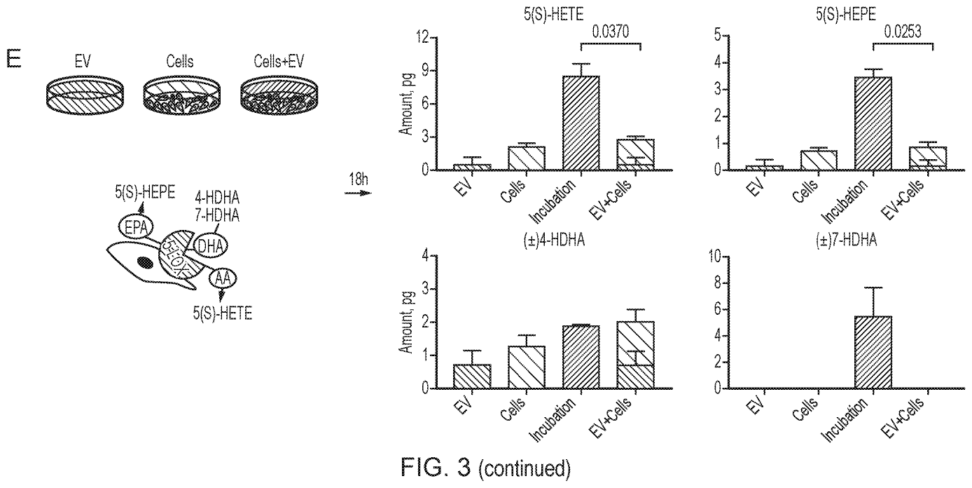

FIG. 3. ACdEV are “active” metabolic compartments
(A) Representative western blots indicate presence of 5-LOX, 15-LOX, 12-LOX and sPLA2 but not COX-2 in ACdEV. (B) Fluorometric lipoxygenase activity assay indicates that ACdEV carry active LOX. (C) Measured decreases in LOX-associated fluorescence in the presence of specific-LOX inhibitors were used to calculate specific activities of 5-LOX, 12-LOX and 15-LOx in ACdEV. (D) LOX activity in ALOX12^{ko} cells incubated for 2 h with EV (Cells+EV, blue bar) was 41% higher than in the cells that were EV-free (Cells, pink bar) indicating that EV can deliver active enzymes after internalisation by host cells. Data was further normalised to the total EV protein amount. (E) Incubation of ALOX5^{ko} cells with 5-LOX-replete ACdEV over 18 h (annotated with Incubation, blue bar) resulted in the significant increase of 5-LOX metabolites 5(S)-HETE and 5(S)-HEPE compared to their cumulative levels in ACdEV and ALOX5^{ko} cells (EV+Cells, yellow/pink bar). Individual contributions of metabolites quantified in ACdEV (EV, yellow bar) and ALOX5^{ko} cells (Cells, pink bar) are represented as separate bars as well. Levels of metabolites represented with Incubation and EV+Cells were statistically compared. All results are expressed as mean±SEM, n=3. Statistics: t-test, two tailed, unpaired.

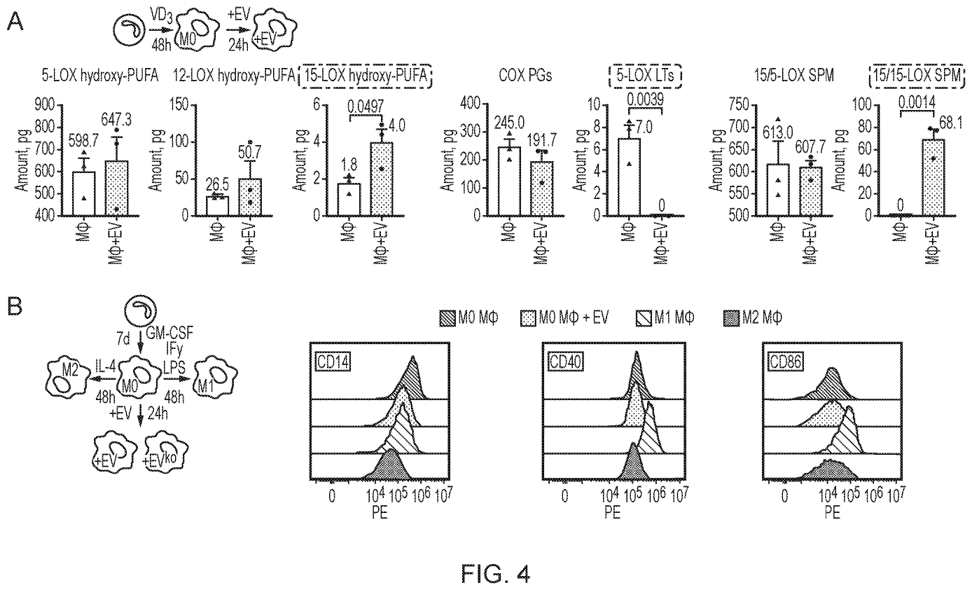

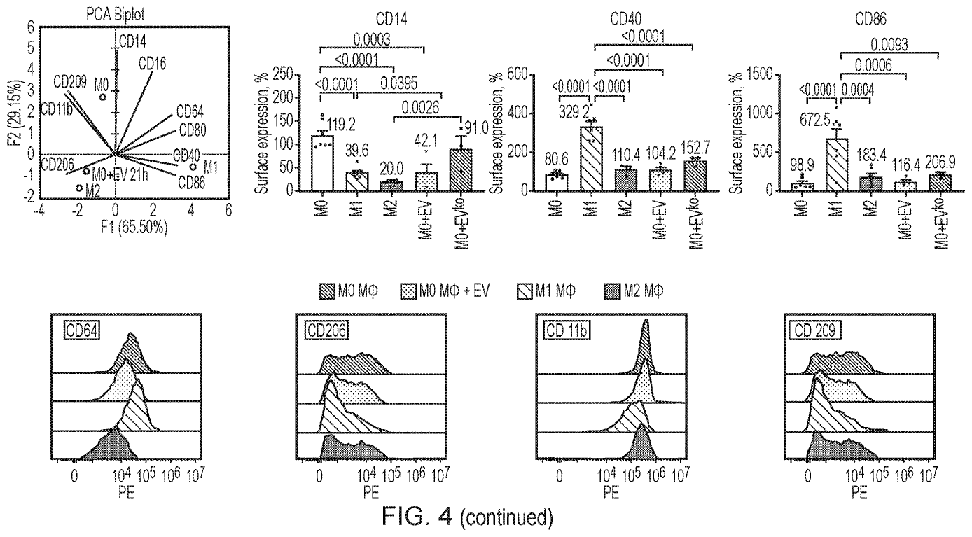

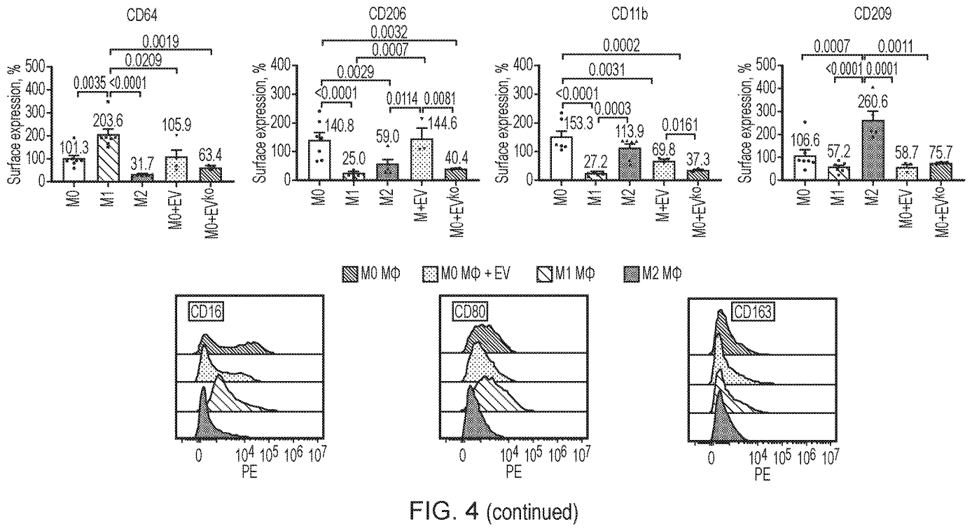

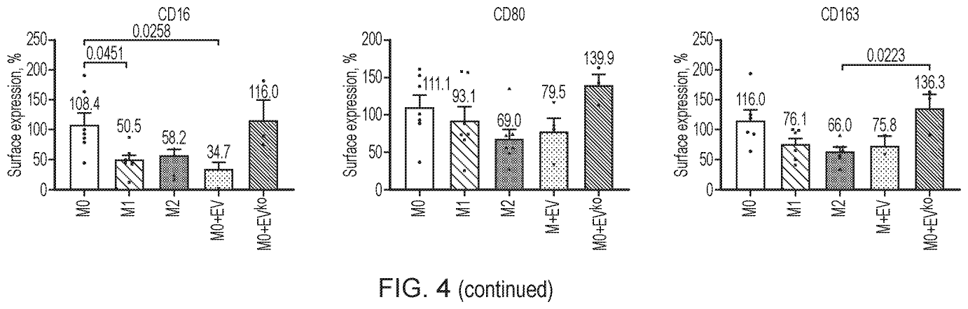

FIG. 4. Acdev Modulate Macrophage Responses

(A) THP-1-derived macrophages (MO, unpolarised, clear bars) were incubated with ACdEV (blue bars) and the indicated lipid mediators assayed. Data suggest upregulation of 15-lipoxygenase (15-LOX) that switches the macrophage phenotype with a significant increase of anti-inflammatory and pro-resolving lipid metabolites (green boxes) derived via 15-LOX oxygenation pathways (15(S)-hydroxy PUFA and SPM produced via double 15-LOX oxygenation of DHA). This is accompanied by the complete disappearance of pro-inflammatory 5-lipoxygenase (5-LOX) derived leukotrienes (LTs) after the vesicle treatment (red box). No changes are observed in the levels of metabolites produced by 12-lipoxygenase and cyclooxygenase. Results are expressed as mean±SEM, n=3, Statistics: t-test, paired, two tailed, *P<0.05.

(B) Primary human monocyte-derived macrophages were analysed by flow cytometry following immunofluorescence staining either as unpolarised macrophages (MO, clear bar), following polarisation to M1 macrophages (red bar), M2 macrophages (green bar) or following treatment with ACdEV (M+EV; blue bar) for 24 h. Surface expression of CD11b, CD14, CD16, CD40, CD64, CD80, CD86, CD163, CD206 and CD209 was monitored using flow cytometry allowed to identify phenotypic differences in three macrophage families: M0—CD14⁺, M1—CD40⁺CD⁶⁴⁺CD86⁺, M2—CD11b⁺CD206⁺CD209⁺. Data was normalised to the mean fluorescence intensities observed for M0 within each replicate and set as 100%. PCA analysis revealed that EV treated macrophages (M0+EV) have a phenotype close to pro-resolving M2. Flow cytometry histograms and PCA biplot are a single biological replicate, representative of at least 3 independent experiments. All results are expressed as mean±SEM. Statistics: one-way ANOVA with Tukey's multiple comparisons test.

FIG. 5: Administration of EV to mice with experimental allergic airways inflammation reduces presence of eosinophils and inflammatory macrophages

Mice were exposed to either PBS (blue bars) as a control or House Dust Mite (HDM) antigen (Red bars) to induce lung inflammation and day 3 after exposure, primary human T cell derived EV were administered locally (i.n.: intra-nasal) or systemically (i.p.: intraperitoneal). After a further 18 hours, mice were sacrificed and lungs were dissociated, and immune cells stained for multicolour flow cytometry. The presence of (A) neutrophils (CD11b⁺/Ly6G⁺), (B) eosinophils (SiglecF+) and (C) inflammatory macrophages MØ (Ly6C⁺) is shown. In each treatment, 6 mice were used across two independent experiments. *P<0.05; **P<0.01. EV in PBS treated mice did not exert any detectable significant effect.

### MATERIALS

RPMI-1640 cell culture medium (phenol red supplemented and phenol red-free', Iscove's Modified'Dulbecco's Medium (IMDM), L-glutamine, penicillin/streptomycin, cycloheximide, Dulbecco's phosphate buffered saline, EDTA, anti-Rabbit IgG (whole molecule)-Peroxidase antibody produced in goat (cat. no. A0545-1ML), anti-Mouse IgG (whole molecule)-Peroxidase antibody produced in rabbit (cat. no. A9044-2ML), docosahexaenoic acid were purchased from Sigma Aldrich (Irvine, UK). Foetal calf serum of South American origin and ACK (Ammonium-Chloride-Potassium) lysis buffer were from Gibco (Thermo Fisher Scientific, UK). Anti-Fas antibody (human activating, CH11 clone) and Amicon® Ultra-15 Centrifugal Filter Unit 10 kDa was from Millipore (Watford, UK). 1α,25-Dihydroxyvitamin D₃ was from Enzo Life Sciences (Exter, UK). Megamix Plus FSC and SSC beads were manufactured by Biocytex (Marseille, France). Acetonitrile and formic acid (both ULC-MS grade) were from Biosolve (Valkenswaard, Netherlands). HPLC grade methanol and ethanol and MS-grade water were purchased from Fisher Scientific (Loughborough, UK). Trypsin Gold, sequencing, was from Promega (Southempton, UK). Recombinant human granulocyte/macrophage colony-stimulating factor (GM-CSF cat. no. 215-GM-010), interleukin-4 (IL-4, cat. no 204-IL-010), interferon-gamma (IFNγ, cat. no. 285-IF-100), BW-B 70C (cat. no. 1304) and PD 146176 (cat. no. 2850) were from R&D systems (Abingdon, UK). Western blotting ECL substrate kit (cat. no. 32109) and pre-stained protein ladder (cat. no. 26619) were from Pierce (Thermo Fisher Scientific, UK). Apoptosis detection kit (cat. no. BMS500FI/300), mouse anti-human CD11b-PE (cat. no. 12-0118-42), mouse anti-human mouse anti-human CD209-PE [eB-h209] (cat. no 17-2099-42), mouse anti-human CD64-PE [10.1] (cat. no 12-0649-42), mouse anti-human CD206-PE (cat. no. 12-2069-42), mouse IgG1kappa isotype control (P3.6.2.8.1) PE (cat. no. 12-4714-42) and lipopolysaccharide (LPS) were from eBioscience (Thermo Fisher Scientific, UK). Mouse anti-human CD86-PE [BU63] (cat. no MHCD8604), mouse anti-human CD80-PE [MEM-233] (cat. no MHCD8004), mouse anti-human CD40-PE [HB14] (cat. no. CD4004), mouse anti-human CD16-PE [3G8] (cat. no. MHCD1604), mouse anti-human CD14-PE [Tük4] (cat. no. MHCD1404), mouse IgG1 isotype control PE (cat. no. MG104), mouse IgG2alpha isotype control PE (cat. no. MG2A04) and BODIPY™ FL N-(2-Aminoethyl)Maleimide (cat. no. B10250) were from Invitrogen (Thermo Fisher Scientific, UK). Laemmli buffer (6× reducing) was from Alfa Aesar (Thermo Fisher Scientific, UK). Urea, thiourea, tris base and Coomassie Brilliant Blue G250 (proteomics grade) were obtained from VWR (Lutterworth, UK). Tween-20 was from Bio-Rad (Watford, UK). Mouse anti-human TSG101 antibody (ab83), rabbit anti-human 5-lipoxygenase (cat. no. ab169755), mouse anti-human 15-lipoxygenase antibody (cat. no. ab119774), rabbit anti-human 12-lipoxygenase (cat. no. ab168384) antibody, rabbit anti-human cyclooxygenase 2 (cat. no. ab151571) antibody and rabbit anti-human phospholipase A2 antibody (cat. no. ab139692) were from Abcam (Cambridge, UK). RosetteSep™ total T cell enrichment cocktail (cat. no. 15061), RosetteSep™ monocyte enrichment cocktail (cat. no. 55018), EasySep™ buffer and Lymphoprep were purchased from Stemcell (Cambridge, UK). Resolvin E1 (cat. no. 10007848), 5(S)-HEPE (cat. no. 32210), 12(S)-HEPE (ca. no. 32550), 15(S)-HEPE (cat. no. 32710), (±) 18-HEPE (cat. no. 32840), 5(S)-HETE (cat. no. 34230), 12(S)-HETE (cat. no. 34570), 15(S)-HETE (cat. no. 34720), Lipoxin A₄ (cat. no. 90410), Lipoxin A₄-d₅ (cat. no. 10007737), Lipoxin B₄ (cat. no. 90420), 5(S)-HETE-d: (cat. no. 334230), Leukotriene B₄ (cat. no. 20110), Leukotriene B₄ -d₄ (cat. no. 320110), Prostaglandin E₂ (cat. no. 14010), Prostaglandin E₂-d₄ (cat. no. 314010), Prostaglandin D₂ (cat. no. 12010), 15(R)-Lipoxin A₄ (cat. no. 90415), Prostaglandin F2α (cat. no. 16010), Prostaglandin F2α-d₄ (cat. no. 316010), 5(S),15(S)-DiHETE (cat. no. 35280), 6-trans-12-epi-Leukotriene B₄ (cat. no. 10012554), Resolvin D₁ (cat. no. 10012554), Resolvin D₂ (cat. no. 10007279), Resolvin D₂-d₅ (cat. no. 11184), Resolvin D₃ (cat. no. 13834), Resolvin D₄ (cat. no. 13835), Maresin 1 (cat. no. 10878), 7-epi-Maresin 1 (cat. no. 13161), (±) 4-HDHA (cat. no. 33200), (±) 7-HDHA (cat. no. 33300), 14(S)-HDHA (cat. no. 15253), 17(S)-HDHA (cat. no. 10009799), 10(S),17(S)-DiHDHA (cat. no. 10008128), arachidonic acid (cat. no. 10007268), arachidonic acid-ds (cat. no. 10007277), eicosapentaenoic acid (cat. no. 90110) and ML 355 (cat. no. 18537) were from Cayman Chemical Company (Ann Arbor, Michigan, USA). Lipoxygenase activity assay kit (cat. no. K978-100) was from BioVision (Milpitas, California, USA). THP-1 cell line was purchased from ATCC (LGC Standards, Teddington, UK). Lipoxygenase CRISPR-Cas9 knockouts HAP1 ALOX12^{ko}, and ALOX5^{ko} and a double knockout ALOX5,15^{ko} cells were engineered by Horizon Discovery (Cambridge, UK). Leukocyte blood cones were purchased from NHS Blood and Transplant (Birmingham, UK).

### Isolation of Primary T Cells and Induction of Apoptosis

Primary human CD3+ T cells were negatively isolated from leukocyte cones (healthy donors). Briefly, the content from one cone (approximately 7 mL) and 3 mL of sterile PBS were mixed and incubated with 0.5 mL of antibody cocktail (RT, 20 min). The volume was adjusted to 20 mL with PBS and layered over 20 mL of Lymphoprep. T cells were separated by 20 min centrifugation at 1,200×g. Isolated T cells were washed with PBS. Cell pellets were resuspended in ACK lysis buffer (5 mL, RT, 5 min) for the removal of red blood cells. Cells were washed twice in PBS and resuspended at a density of 4×10⁶ cells/mL in serum-free RPMI 1640 medium (phenol red-free, supplemented with antibiotics). From one blood cone it was possible to isolate 2.5-3×10⁸ total T cells.

### Induction of Apoptosis

Apoptosis was of primary T cells and HAP1 ALOX5,15^{ko} cells was induced with anti-Fas antibody (1:10000 dilution) and cycloheximide (20 μg/mL) under serum-free conditions over 15 h and 12 h respectively. The degree of cell death (apoptosis vs secondary necrosis) was assessed using flow cytometry (Cytoflex S, Beckman Coulter, High Wycombe, UK) using annexin V-FITC and propidium iodide staining.

### Isolation of EV

Extracellular vesicles (EV) were isolated from the supernatants of apoptosis-induced serum-free cell cultures. Briefly, apoptotic cells and cellular debris were pelleted (20 min at 2,000×g) and remaining apoptotic secretome was concentrated on spin columns to a final volume below 1.5 mL. EV were separated from soluble proteins using pre-packed size exclusion chromatography columns (qEV original, Izon Science, Oxford, UK). The columns were preconditioned and eluted with PBS. The first 3 ml of eluate was discarded whilst the subsequent 3.5 mL of eluate was EV-rich, with minimal to no soluble protein contamination.

For the purpose of Western blotting and proteomic profiling of EV, 3.5 mL of EV isolate was further concentrated to a volume below 150 μL (containing one blood cone isolate equivalent) to ensure high EV density. For the purpose of lipoxygenase activity assays, the final EV volume was kept under 100 μL, and EV were used immediately for downstream analysis. EV separation on qEV columns was performed with serum-free RPMI when EV were to be used for further co-incubations with monocyte-derived macrophages.

EV protein concentration was estimated using a Bradford assay. Isolated EV were then stored at −20° C. until analysis.

### Measurement of EV Size and Concentration

Size and concentration of EV was measured from the final EV isolates prior to storage, or used for different assays. Size distribution of EV and their concentration was measured in PBS using Tuneable Resistive Pulse Sensing (TRPS) technology on a qNano Gold (Izon Science, Oxford, UK) particle analyser with 150 nm nanopore. The machine was calibrated with silica beads of 200 nm. For the optimal measurement accuracy, 500 events were recorded for each measurement.

### Flow Cytometry of EV

Bodipy dye linked to a maleimide probe (1 μM final concentration) was added to the 2000×g apoptotic T cell supernatant and incubated on ice for 4 h to allow EV staining. For annexin V staining of EV, 50 μL of 2000×g supernatant was diluted 10 fold in ice-cold annexin V binding buffer and probed with 5 μL of annexin V-FITC prior to measurement.

MegamixPlus beads were used to adjust the cytometer settings to enable confident detection of particles larger than 150 nm. Detection of small particles was triggered with violet SSC (manual cut-off for area of 149713) and the manual cut-off, applying the following gains: FSC-101, SSC-74, VSSC-1801, FITC-141). Sample was infused with the flow rate of 10 μL/min until 50,000 events were collected.

### Label-Free Relative Quantification of Proteins by LC-MS/MS

Proteins in apoptotic T cell lysates and ACdEV isolates (5 replicates, 30 μg each) were reduced in Laemmli buffer for 15 min at 65° C., separated by molecular weight on 10% SDS-PAGE and stained with Coomassie G 250 blue (0.5% w/v in 40% aqueous methanol and 10% glacial acetic acid) for 4 h. After destaining, each sample lane was divided into five bands of the same size across all samples on a gel. After gel sections were excised, transferred into polypropylene tube and diced, proteins were destained in 50% acetonitrile in 50 mM ammonium bicarbonate. After complete destaining, diced gels were dehydrated with pure acetonitrile and vacuum dried for 30 minutes minimum in a vacuum concentrator (Eppendorf, UK). Gels were rehydrated with trypsin solution in 6 mM ammonium bicarbonate (25:1 protein to trypsin ratio) and allowed for the protein digestion overnight with shaking (700×g, 37° C.). Peptides were extracted sequentially from diced gel using 30%, 50% and pure acetonitrile for 15 min in an ultrasonic bath. Extracts from a single sample section were combined into one tube, vacuum dried and stored at −20° C. prior to analysis.

Samples were reconstituted in 100 μL of 3% aqueous acetonitrile and 0.1% formic acid for liquid chromatography-coupled tandem mass spectrometry (LC-MS/MS) analysis. Peptides were separated and analysed using an nUPLC system (Acquity M class, Waters, UK) coupled to 5600 TripleTof (AB Sciex, UK) operating in information dependent (IDA) mode. Using a single pump trapping mode (5 μL/min, 1 min, 1% acetonitrile in aqueous 0.1% formic acid), peptide solution (5 μL, ~15 pmol) was injected onto a trap column (nanoEase M/Z Symmetry C18 Trap Column, 100 Å, 5 μm, 180 μm×20 mm, Waters, UK), and separated on the analytical column (viper fitting PepMap™, C18, 5 μm, 100 Å, 300 μm×1 mm, ThermoScientific, UK) using 1% of eluent B (acetonitrile in aqueous 0.1% formic acid) at a flow rate of 15 μL/min. Peptides were subsequently separated on an analytical column (Acclaim™, PepMap™ C18, 3 μm, 100 Å, 75 μm×150 mm, ThermoScientific, UK) with the following gradient: 0-45 min 1-45% B, 45-49 min 45-90% B, 49-52 min 90% B, 52-67 min 1% B. Stable electrospray was formed at 2200 V using a PicoTip™ emitter (New Objective, Germany). The 10 most intense ions from each high-resolution MS survey scan were selected for high sensitivity MS/MS, while acquired ions were temporarily excluded from MS/MS acquisition for 30 s. The mass spectrometer was calibrated prior to acquisition to ensure a high mass accuracy on both MS and tandem mass spectrometry (MS/MS) levels.

Relative quantification was undertaken using Progenesis QI for proteomics software (version 4, Nonlinear Dynamics, UK) multi fraction setup. Only protein-unique peptides were used for relative quantification. MS/MS data were searched using Mascot Daemon (ver 2.5) against the curated SwissProt database, with the following search restriction parameters: mass tolerance of 0.1 Da for MS and 0.6 Da for MS/MS spectra, a maximum of 2 trypsin missed-cleavages, Homo sapiens taxonomy, variable modifications of methionine oxidation and cysteine carbamidomethylation.

Gene ontology (GO) data analysis was done using PANTHER (Protein Analysis Through Evolutionary Relationships) classification system against Homo sapiens whole genome list available from Mar. 23, 2020. Analysis was done using statistical overrepresentation test with Bonferroni correction for multiple testing.

### Detection of Lipid Metabolites and Pufa by Targeted Mass spectrometry

For the metabolome analysis EV were collected and purified from approximately 1×10⁹ primary T cells. Samples were spiked with 1 ng of isotopically-labelled internal standards kept on ice for 10 min, then treated with 5 volumes of ice-cold methanol and kept on ice for 10 min with occasional vortexing to allow for the extraction of lipid metabolites. Lipid metabolites were enriched on solid phase extraction cartridges (Oasis HLB, 10 mg, Waters, Manchester, UK) previously wetted with methanol (200 μL) and equilibrated with 15% aqueous methanol acidified with 0.1% formic acid (200 μL). Sample (acidified in 15% methanol) was loaded and cartridge washed with equilibration buffer and hexane (600 μL). Lipid metabolites were eluted with 20% methanol in butyl-acetate (2×1 mL) and vacuum dried. Samples were stored at −20° C. prior analysis.

Optimization of MS acquisition conditions was performed on ESI-QqLIT-MS (QTRAP 5500, AB Sciex, Warrington, UK) operated in a negative ion mode with an ionization voltage of −4.5 kV, entrance potential of −10 V, and ion source temperature of 400° C. Solutions of authentic and internal standards (500 pg/μL in 0.1% formic acid in 50% aqueous methanol) were used for the optimization of normalized collision energy (CE), declustering potential (DP), and exit quadrupole potential (CXP) for each Q1/Q3 transition. Using the syringe pump (Harvard Apparatus GmbH, March-Hugstetten, Germany) with 20 μL/min flow rate, standard solutions were directly infused into mass spectrometer for the optimization of Q1/Q3 transition pairs. The final time scheduled MRM included 88 Q1/Q3 transition pairs, with up to four most intense structure specific transitions for each analyte.

The LC-MS/MS(MRM) was performed on a Dionex Ultimate 3000 RS UPLC (Thermo Fisher Scientific, UK) system ESI-QqLIT-MS. Samples were separated on the XSelect HSS T3C18 -column (particle size 2.5 μm, 2.1×10 mm, pore size 100 Å) using a column temperature of 50° C. and flow rate of 200 μL/min. Samples (50 μL) were infused using 50% methanol in 0.1 aqueous % formic acid. Analytes were chromatographically separated mixing the eluent A (0.1% formic acid in water) and B (0.1% formic acid in methanol) as following: 50% B for 5 min, 50-90% B in 40 min, 90-100% B in 5 min, 100% B for 4 min, 100-50% B in 1 min, 50% B in 11 min. Quantification of analytes in the samples was achieved after their normalization to an internal standard and against the corresponding calibration curves for the same Q1/Q3 transition pair. All authentic standards were measured using a final acquisition method for a range of concentrations between 500 fg to 750 pg to allow for a design of individual calibration curves with the best linear fit. Data was processed using Analyst Software (version 1.6.2, AB Sciex).

### Western Blotting

Following protein amounts were used for a detection of EV proteins: 30 μg for TSG101; 50 μg for LOX-5, LOX-15, LOX-12; COX-2 and PLA₂. EV samples were reduced in Laemmli buffer (15 min at 65° C.) and proteins resolved using 10% SDS-PAGE. Following transfer to nitrocellulose membrane (Amersham™ Protran™ Premium NC Membrane, GE Healthcare), membranes were blocked in 5% milk in TBS-T on an orbital shaker (1 h or overnight at 4° C.), and probed with primary monoclonal antibody (1:1000 dilution). After three washes in TBS-T, membrane was incubated with HRP-conjugated polyclonal antibody (anti-mouse or anti-rabbit, 1:5000 dilution). Using ECL substrate, labelled proteins were detected on a G: BOX XT4 (Syngene, Cambridge, UK).

### Lox Activity Assay

For LOX activity assay, EV were collected from apoptotic cultures of primary human T cells as described above. LOX activity assay was performed using a fluorimetric assay kit following the manufacturer's guideline. Fluorescence was recorded on a SpectraMax Gemini EM Microplate Reader (Molecular Devices, Reading, UK) in kinetic mode, measuring fluorescence at 500/536 nm (excitation/emission) every 5 minutes up to maximum 90 minutes. Individual activity contributions of 5-LOX, 12-LOX and 15-LOX were addressed using their corresponding inhibitors BW-B 70C, ML355 and PD146176, respectively. Inhibitors were prepared daily in DMSO up to their maximal soluble concentrations.

### Transfer of Lox Activity

Transfer of LOX activity from EV to cells was measured after 2 h incubation of freshly isolated EV (derived from 7×10⁷ T cells, 170 μg protein) with CRISPR/Cas9 ALOX12^{ko} cells. Synthesis of 5(S)-hydroxy-PUFA was measured in CRISPR/Cas 9 ALOX5 knockout cells (2×10⁶ cells) after 18 h incubation with freshly prepared EV. Cells were plated into a 6-well plate at full confluence. Freshly prepared EV (corresponding to 4×10⁷ T cells) were incubated with adherent cells in IMDM serum-free medium. As a control, EV-free cells were plated and treated under the same conditions. Cells were harvested by scraping and centrifugation (5 min, 300×g).

For the LOX activity assay, pelleted cells were lysed in LOX buffer (provided in the LOX assay kit). Protein concentration was estimated using Bradford assay. Samples were immediately used for the LOX assay. For the lipid metabolomics profiling of 5-LOX-derived LM, enrichment and detection of LM in EV, cells and corresponding supernatants was done in parallel as described above.

### Human Macrophage Phenotyping

Human THP-1 monocytic cells were cultured routinely in RPMI-1640 medium supplemented with 10% foetal calf serum and 2 mM L-glutamine, 100 U/mL penicillin and 100 μg/mL streptomycin. As required, differentiation to THP-1-derived macrophages (MO) was induced with 100 nM VD₃ over 48 h with an initial cell density of 5×10⁵ cells/mL. Differentiated cells were washed and resuspended in the serum-free medium at 1×10⁶ cells/ml and incubated as appropriate with freshly prepared ACdEV (collected from 4×10⁷ apoptotic T cells) in a 6-well plate for 24 h at 37° C. As a control, differentiated cells were incubated under the same conditions without ACdEV. Cells were collected from the plate, pelleted at 300×g and lipid metabolites were enriched and analysed as described above.

For primary human monocyte-derived macrophages, a single leukocyte cone (healthy donors) was volume adjusted to 10 ml with PBS in a sterile polypropylene tube with 1 mM EDTA. Human monocytes were negatively isolated by incubating the blood sample with 0.5 mL of monocyte-enrichment cocktail (20 min, RT), diluting the blood with 10 mL of EasySep buffer and separating it on a Lymphoprep gradient (1200×g, 20 min). Isolated monocytes were washed with EasySep buffer and residual red cells removed by resuspending in RCK lysis buffer (5 mL, RT, 5 min). Cells were washed twice in EasySep buffer, resuspended in a serum-free RPMI 1640 medium (phenol red-free, supplemented with antibiotics) at a density of 3×10⁶ cells/mL and seeded into 75 cm² flasks. From one leukocyte cone it was possible to isolate over 1×10⁸ monocytes.

Human monocytes were differentiated into monocyte-derived macrophages over 7 days with 20 ng/ml of GM-CSF. Cells were carefully collected and washed in PBS. Monocyte-derived MO macrophages (2.5×10⁶ cells/ml) were stimulated for 48 hours with 20 ng/ml IFN-γ and 1 ng/ml LPS to promote M1 phenotype, while M2 phenotype was stimulated with 20 ng/ml IL-4. The ability of EV to induce monocyte-derived M0 macrophage polarisation towards M1 or M2 phenotype was tested upon 24 h incubation with EV derived from 3×10⁸ apoptotic T cells. Phenotypic fingerprinting of the human monocyte-derived macrophages was assessed by flow cytometry using a panel of fluorescent antibodies: CD14, CD16, CD40, CD80, CD86, CD11b, CD64, CD206, CD209 and corresponding isotype controls.

### Results

### ACdEV Are Released From Apoptotic Cells

The focus of this study was to investigate how immune cells during early apoptosis communicate their presence to the immune system via ACdEV release, and whether these vesicles have immunomodulatory properties. Our previous work shows that small ACdEV (<1 μm) within 2000×g supernatants promote recruitment of macrophages¹⁷. The present study focused on these small EV which, due to their size, may be capable of transmission over longer distances in vivo. To this end, ACdEV were prepared from freshly isolated primary human CD3⁺ T cells that were immediately committed to apoptosis with a combination of agonistic anti-Fas antibody in the presence of cycloheximide. Flow cytometric monitoring of apoptosis with annexin V/propidium iodide staining reveals that the maximal levels of apoptosis with minimal secondary necrosis were reached 15 h post-induction. It is notable that this timeframe with primary cells was longer than typical in cell lines²⁰. Following established guidelines in the field²¹, ACdEV were isolated from serum-free supernatants after differential centrifugation to remove apoptotic cells, cell debris and larger apoptotic bodies. Finally, ACdEV of high purity were isolated using size exclusion chromatography. Measurement of ACdEV size and concentration reveals that over the course of apoptosis a T cell releases ACdEV with a modal size of 110 nm (FIG. 1A), equating to, on average 400-600 ACdEV per cell. Healthy cells released considerably lower EV numbers, which did not reach the TRPS counts for the quality data processing (>500 events per sample). The broad size range of 60-720 nm indicates that apoptotic T cells produce heterogeneous ACdEV population, likely composed of a mixture of exosomes and microvesicles. Indeed, western blot against exosome marker TSG101 indicated that at least a portion of isolated ACdEV is likely of exosomal origin (FIG. 1A, inset). In addition, we have we confirmed vesicle presence by flow cytometry after fluorescent labelling with thiol-reactive Bodipy dye (FIG. 1B).

Exposure of phosphatidylserine (PS) on the outer leaflet of the plasma membrane during apoptosis facilitates recognition and uptake of apoptotic cells by phagocytes^{22,23}. It therefore seemed likely that ACdEV may also have exposed PS on their surface. Labelling of ACdEV with annexin V revealed that around 69% of ACdEV carry have PS exposed on their surface (FIG. 1C). Given that PS on apoptotic cells modulates immune responses by promoting uptake of apoptotic cells²⁴, and that ACdEV drive production of TGF-β1²⁵, it possible that PS may be an “active” component of ACdEV, independent of the cell type from which they are generated.

In order to capture a full picture of the composition of ACdEV, we investigated if there is a preferential enrichment of proteins into ACdEV during apoptosis and what proteomic fingerprint ACdEV might have. Using a bottom-up proteomics approach and label-free quantification, we compared the proteomes of apoptotic T cells and the ACdEV they released during apoptosis. We were able to identify 1114 proteins overall, while 865 qualified for the relative quantification based on the inclusion/exclusion criteria At the time of publication, the extracellular vesicle database Vesiclepedia 26 identifies 2676 proteins related to all types of EV derived from T cells. Our ACdEV proteome overlaps with 608 proteins, while 260 proteins were identified in our T cell-derived extracellular vesicles for the first time, and 19 proteins of those associated with extracellular vesicles of any cellular origin (FIG. 1D).

Here, we compared proteomes of apoptotic T cells and ACdEV, and we found that ACdEV are significantly enriched (P<0.01) in 406 proteins (FIG. 1E). Of these, 10 proteins were present exclusively on ACdEV. Those 406 proteins were further categorised using Gene Ontology (GO) enrichment for biological processes, cellular components and molecular functions (FIG. 1F). As expected, the greatest protein enrichment within ACdEV was noted for those associated with the gene ontology process “extracellular exosome” and “vesicle”, further confirming efficient ACdEV isolation. Specifically, we identified enrichment in the proteins from the annexin family, HSP90, 14-3-3 member proteins, histones H1, Rabs, GTPases, flotilin 1 and 2, tetraspanins and T cell-specific receptors, including members of HLA I and HLA II, all commonly associated with extracellular vesicles²⁷ (FIG. 1G). Importantly, biological processes related not only to “vesicle-mediated transport”, “exocytosis”, “mRNA metabolism”, but also adhesion, leukocyte activation, immune responses, MHC I class antigen presentation, upregulation of cell migration and wound healing.

Closer inspection of identified proteins revealed that ACdEV are preferentially enriched in the number of proteins that allow them to communicate their presence to the immune system and ensure their uptake. Well-known “eat-me” signals such as annexin A1²⁸ and calreticulin²⁹ were found on ACdEV; while annexin A1 was 5.2-fold higher in ACdEV, calreticulin was found mainly on apoptotic T cells (8.1-fold). Interestingly, “do-not-eat-me” signals like CD47³⁰ (3.1-fold) and CD31³¹ (2.5-fold) were also more abundant in ACdEV. Additionally, proteins that regulate vesicle uptake via clathrin-dependent endocytosis, like clathrin heavy chain 1³²(CLH1 , 7.0-fold), clathrin-independent endocytosis, like transforming protein RhoA³³ (2.3-fold) and Ras-related C3 botulinum toxin substrate 1³³ (Rac1 , 2.2-fold), and adhesion molecules, such as CD44³⁴ (3.8-fold), integrin alpha L (4.9-fold) and integrin beta 2 (3.0-fold), including ICAM-3¹⁷ (5.4-fold), were also enriched in ACdEV compared to apoptotic T cells.

Apart from the uptake machinery, ACdEV are enriched in proteins such as CD48 (2.8-fold), CD266 and SLAMF6 (both exclusive to ACdEV) that may represent a mechanism by which ACdEV communicate with lymphocytes. By ligation or association with their receptors CD244 and LFA-1 or self-ligation, CD48, CD266 and SLAMF6 were demonstrated to interact with T cells and NK cells, leading to their activation^{35,36,37}. It is worth mentioning that ACdEV were significantly enriched with cell receptors like GP 183 (also known as EBI2, 458-fold increase) and SLAMF6 (exclusive to ACdEV). However, the role of these proteins on EV was not investigated up to date.

Furthermore, we found that proteins with previously described immunomodulatory properties, such as interleukin-16 (IL-16)³⁸ and macrophage migration inhibitory factor (MIF)³⁹ are present on ACdEV. Finally, arachidonate 5-lipoxygenase-activating protein (AL5AP, also known as FLAP) and leukotriene A-4 hydrolase (LKHA4), both involved in the synthesis of pro-inflammatory leukotrienes were found in ACdEV.

Interestingly, ACdEV were highly enriched in ATP binding-cassette proteins (ABC transporters) such as multidrug resistance protein 1 (MDR1, 13.3-fold) and multidrug resistance-associated protein 1 (MRP1, exclusive to ACdEV), also known as ABCB1 and ABCC1 transporters. Although data on ABC transporters and EV is limited, they were reported to serve as efflux pump on drug-loaded cancer-cell-derived EV that support cancer drug resistance^{40,41}.

### ACdEV Carry Lipid Metabolites

Lipid mediators of inflammation (LM) are actively produced and released by immune cells during an inflammatory response. While immunomodulatory properties of ACdEV have been observed previously^{15,16,17,42,43,44,45,46}, little is known of their functionally active components, and especially of their bioactive lipid factors. To address this specifically, we undertook lipid metabolome profiling of ACdEV to identify their LM signature. Our lipid metabolomic MRM (multiple reactions monitoring)-based platform was finely tuned to target a range of 32 pro-inflammatory and dual-acting SPM derived from AA, eicosapentaenoic acid (EPA) and docosahexaenoic acid (DHA) via the LOX and COX pathways (FIG. 2A) . For each analyte, a set of structure specific Q1/Q3 (parent ion to daughter ion) “transition pairs” and a characteristic elution time were established using authentic standards. For a positive identification it was necessary that spectral characteristics (parent ion and daughter ions) and the retention time of analyte and its standard matched. Using established criteria, we identified and quantified presence of 16 LM and their poly-unsaturated fatty acid (PUFA) precursors in ACdEV samples (FIG. 2). Furthermore, we detected 11 additional compounds at LM-specific Q1/Q3 transition pairs but without matching retention times, indicating detection of an isobaric analyte with LM-overlapping spectral properties. It is likely that these unknown analytes are the positional or stereo-isomers of the targeted LM, therefore we annotated them with asterisks (e.g. LM*, LM** etc.) and included them in our report (Figure S2).

Our results indicate that ACdEV are loaded with free ω-6 and ω-3 PUFA, mainly AA and DHA, with 35-45-fold lower levels of EPA (FIG. 2B). Lipoxygenases catalyse PUFA oxygenation to PUFA-hydroperoxides which are reduced rapidly to form stable hydroxy-PUFA^{47,48}. These early LOX metabolites can then serve as substrates for the further LOX-mediated oxygenations to form a range of LM including SPM. We demonstrate that during T cell apoptosis, all PUFA (AA, DHA, EPA) are used as substrates to produce hydroxy-PUFA by the action of 5-, 12- and 15-LOX and these are packed into ACdEV (FIG. 2C). Furthermore, 12-LOX metabolites such as 12(S)-HETE, 12(S)-HEPE and 14(S)-HDHA were found to be the major hydroxy derivatives of AA, EPA and DHA respectively.

In defining LM carriage within ACdEV, it is notable that we identify significant carriage of pro-inflammatory prostaglandins, such as PGE₂, PGD₂, PGF_{2α} LTB₄ and its aspirin analogue 6-trans-12-epi-LTB₄, with PGE₂ being the most abundant amongst them (FIG. 2D). It was possible to additionally detect presence of one PGF_{2α} analogue −PGF_{2α}*, and four analogues of LTB₄. In fact, PGF_{2α}* appears to be the most abundant pro-inflammatory LM in ACdEV, while levels of both LTB₄*** and LTB₄*** were greater than of LTB₄.

Dual-acting SPM (i.e. those with pro-and anti-inflammatory functions) such as lipoxins, resolvins, maresins and protectins, were not detected in ACdEV. However, we detected five SPM analogues (SPM*) suggesting that diversity of ACdEV-bound SPM is different from that anticipated (FIG. 2E). The unidentified SPM analogues most likely correspond to the family of AA-derived lipoxins (LXB₄*, LXB₄**) and DHA-derived resolvins (RvD₃*, RvD₄*) and protectins (PDX*). SPM are a relatively young class of biomolecules, and it is reasonable to assume that biodiversity of SPM is greater than what scientific understanding currently has identified.

Temporal biosynthesis of SPM and an overall balance of pro-inflammatory LM to pro-resolving LM and SPM drives inflammation or contributes to its resolution. Hence, we assessed the combined levels of all pro-inflammatory LM including PG* and LX*, and compared them with AA-derived SPM* and total SPM* (FIG. 2F). Our findings indicate that during T cell apoptosis, ACdEV are secreted with a distinctive pro-resolving LM signature, contributed mainly by the AA-derived SPM from the lipoxin family.

### ACdEV Represent an Active Metabolic Compartment

Following our identification of ACdEV as extracellular compartments rich in bioactive LM, with a pro-resolving phenotype, it was a priority to establish the enzyme-active nature of ACdEV. Recently reported carriage of enzymes in EV^{49,50,51} supports further our hypothesis that ACdEV are ‘active EV’⁵² in that they also carry enzymes responsible for the production of LM. Indeed, western blot analysis for 5-LOX, 15-LOX, 12-LOX and secretory phospholipase A₂ (PLA₂) showed that ACdEV carry these components of the machinery for the production of leukotrienes and SPM although COX-2, responsible for the synthesis of pro-inflammatory prostaglandins, was not detected by western blot (FIG. 3A).

From these initial observations, we further investigated whether ACdEV-associated LOX enzymes are in their active form. To test if the LOX are present in the ACdEV as active enzyme(s), we used a fluorometric assay where LOX action on the substrate would form an intermediate sensitive to the presence of LOX probe, resulting in a fluorescence signal (excited at 500 nm, measured at 536 nm). Measuring LOX activity in ACdEV over time produced a signal significantly in excess of those from the positive control, and the signal-specificity to LOX was confirmed by abolition of the signal to baseline levels in the presence of enzyme inhibitor (FIG. 3B). Using an oxidized probe standard, we designed a calibration curve covering 0 and 10 pmol/well, which allowed us to calculate LOX activities in ACdEV (FIG. 3C). We found that overall specific LOX activity in ACdEV is 2.44 mUnits mg⁻¹ mL⁻¹. Specific activities of 5-LOX, 12-LOX and 15-LOX isoforms were confirmed through the incorporation of isoform specific inhibitors. We show that measured LOX activity in ACdEV from primary human T cells is dominated by the 15-LOX (2.34 mUnits mg⁻¹ mL⁻¹), while 12-LOX (1.43 mUnits mg⁻¹ mL⁻¹) and 5-LOX (1.21 mUnits mg⁻¹ mL⁻¹) activities were of similar but lower values.

The immunomodulatory properties of EV are often attributed to the ligation and activation of so-far ill-defined surface receptors on recipient immune cells⁵³. However, as extracellular vesicles are often internalised by recipient cells, delivery of the EV-bound cargo is also likely to have a significant impact on the recipient cells and their phenotype. Therefore, we investigated if LOX-bearing ACdEV can deliver active enzymes to the recipient cells. To analyse this specifically, LOX-deficient cells were incubated with ACdEV and after incubation, enzyme activity and production of LOX-specific metabolites in the cells was assessed. The recipient haploid cells endogenously express negligible levels of ALOX5 (0.21 transcripts per million, TPM) and ALOX15 (0.06 TPM) genes and very low levels of ALOX12 (1.07 TPM). However, to control for this borderline LOX activity in the recipient cells, we used ALOX12^{ko} (knockout) cells and incubated them with the freshly isolated ACdEV for 2 h. This time frame was selected as it should allow for considerable ACdEV uptake without significant loss of ACdEV-associated enzyme activity. As a control, ALOX12^{ko} cells that were not treated with ACdEV were measured to assess any, limited, endogenous LOX activity. Our results showed 41% higher LOX activity in the cells incubated with ACdEV indicating that LOX remains active in the host cells after ACdEV uptake (FIG. 3D).

The most studied human LOX is 5-lipoxygenase (5-LOX). It preferentially oxygenates ω-6 and ω-3 PUFA at the “S” face of the C-7 carbon to convert them to its 5(S)-hydroperoxy analogues, further reduced to 5(S)-hydroxy-PUFA⁵⁴. In fact, the discovery of 5-LOX dates back to 1976 when the novel AA metabolite 5(S)-HETE was detected in neutrophils⁵⁵. To investigate the biosynthesis of 5-LOX-specific IM from ACdEV-derived enzyme, we incubated ACdEV for 18 h with ALOX5^{ko} cells, and monitored levels of 5-hydroxy-PUFA across different experimental conditions. We had already established that 5(S)-HETE, and 5(S)-HEPE were some of the most abundant LM present in ACdEV (FIG. 2C), and this posed a technical challenge for confident detection of newly synthesised 5-LOX product(s) in the cells following enzyme transfer from ACdEV, as opposed to detection of ready-formed LM carried in the EV. To control for this, we used significantly reduced doses of vesicles (25-fold lower vesicle dose than for LM profiling). This resulted in levels of 5(S)-hETE in ACdEV to be as low as 1.24 pg while 5(S)-HEPE was below the detection limit. Incubation of ACdEV (5-LOX replete) with ALOX5^{ko} cells resulted in the significant increase of 5-LOX products: 5(S)-HETE (−EV: 1.98 pg, +EV: 9.62 pg) and 5(S)-HEPE (−EV: 0.53 pg, +EV: 3.19 pg). These levels in the knockout cells (annotated ‘Incubation’ in FIG. 3E) were significantly higher than the sum of these metabolites in the ACdEV and control cells (annotated EV+Cells in FIG. 3E).

In the absence of appropriate analytical standards with “S” stereo centres, we measured (±)4-HDHA and (±)7-HDHA, metabolites of DHA. While we observed no significant increase in the levels of (±) 4-HDHA in the cells (−EV: 1.29pg, +EV: 3.22 pg) compared to ACdEV (1.24 pg), (+)7-HDHA was detected only after the incubation of ALOX 5ko cells with ACdEV (−EV: not detected, +EV: 7.68 pg). Although both are primary 5-LOX metabolites, these data suggest that 7-HDHA is a more dominant product of 5-LOX metabolism of DHA. Overall, our results indicate that ACdEV can deliver active LOX to the cells, which in turn results in the endogenous synthesis of 5-LOX-derived metabolites. Thus, transfer of active enzymes by ACdEV during leukocyte apoptosis could represent a route by which ACdEV modulate immune responses. These results also raise the possibility that EV may help to modulate activity in non-immune (perhaps non-professional phagocytes) recipient cells.

### ACdEV Control Macrophage Phenotype

Macrophages (MΦ) are considered elite innate immune effector cells because of their professional phagocytic, regulatory and repair functions. In response to endogenous and exogenous stimuli macrophages display remarkable plasticity, enabling changes to their physiology including expression of surface proteins, gene signatures and production of inflammatory mediators⁵⁶. This plasticity is central to their function in the control of inflammation. Here, we demonstrate that ACdEV released during T cell apoptosis have a distinctive pro-resolving phenotype, with higher levels of dual acting analogues SPM* compared to pro-inflammatory leukotrienes (LTs) and prostaglandins (PGs), and carriage of active lipoxygenases with the highest specific activity of 15-LOX. Moreover, we show that ACdEV can deliver active enzymes to the host cells which continue to oxygenate non-esterified PUFA. To investigate whether delivery of these ACdEV can induce a change in macrophage phenotype, we monitored the production of lipid mediators of inflammation (LM) and the change of surface markers in monocyte-derived macrophages incubated with ACdEV.

Using established our lipid metabolomic platform we monitored LM profiles in 1α,25-dihydroxy vitamin D₃ (VD₃) THP-1-derived macrophages (considered here as MO macrophages) before and after 24 h incubation with ACdEV. In response to vesicle uptake, unpolarised macrophages (MO) may change their phenotype towards pro-inflammatory (M1) or pro-repair (M2), which would alter their LM profile. Distinct LM signatures have been described for these macrophage polarisations⁵⁷. Whilst pro-inflammatory M1 macrophages produce higher levels of PGs and LTs, higher levels of SPM-like resolvins (RVs) and lipoxins (LXs) were characteristic for pro-repair M2 macrophages⁵⁷.

Overall, we quantified 16 lipid metabolites and three PUFA (see Figure S3 for individual levels) in THP-1-derived macrophages. Incubation of ACdEV with MΦ macrophages did not induce change in the cumulative levels of hydroxy-PUFA formed via 5-LOX pathway (MΦ: 598.7 pg, MΦ+EV: 647.3 pg) or the 12-LOX pathway (M101 : 26.5 pg, MΦ+EV: 50.7 pg), though levels of 15-LOX-derived hydroxy-PUFA (MΦ: 1.8 pg, MΦ+EV: 4.0 pg) were significantly higher (FIG. 4A). Total levels of pro-inflammatory prostaglandins (MΦ: 245.0 pg, MΦEV: 191.7 g), whilst not significantly different, suggested a decrease in levels after vesicle uptake, while 5-LOX-derived pro-inflammatory leukotrienes (MΦ: 7.0 pg, MΦEV: nd) were no longer detected. Finally, treatment of macrophages with ACdEV did not influence the total levels of 15-/5-LOX-derived SPM (M+: 613.0 pg, MΦ+EV: 607.7 pg), though notably ACdEV supported de novo synthesis of 15-/15-LOX SPM (MΦ: nd, M++EV: 68.1 pg). Taken together, our results indicate that incubation of ACdEV with THP-1-derived macrophages leads to macrophage upregulation of 15-LOX synthesis of anti-inflammatory, pro-resolving lipid mediators such as 15(S)-hydroxy-PUFA and protectin D1, whilst “switching off” 5-LOX synthesis of pro-inflammatory leukotrienes (LTB4/LTB4*). These observed changes in the LM profiles upon ACdEV exposure are characteristic for the LM “class switch” and macrophage phenotype change towards pro-resolving (M2).

In order to assess the ability of these ACdEV to change phenotype within a primary human macrophage system, surface immunophenotyping was undertaken in human monocyte-derived Mo in the presence or absence of ACdEV treatment. Human primary monocytes were isolated from leukocyte cones and stimulated them with GM-CSF to produce monocyte-derived macrophages (unpolarised, MO). The cells were further stimulated either with LPS and IFNγ to produce ‘classically-activated’ macrophages (pro-inflammatory, M1), or with IL4 to generate ‘alternatively-activated’ macrophages (pro-repair, M2). Surface expression of CD11b, CD14, CD16,CD40, CD64, CD80, CD86, CD163, CD206 and CD209 in M0, M1 and M2macrophages was monitored by flow cytometry. Monocyte differentiation antigen CD14 was highest in M0 MØ and this was significantly downregulated following polarisation to M1 or M2 and by treatment with ACdEV, clearly demonstrating ACdEV activity in this assay (FIG. 4B). Classically-activated M1 macrophages are best characterised with the high surface expression of CD40, CD86 and CD64, with CD40showing the most robust upregulation. Alternatively-activated M2 macrophages show high surface expression of CD11b, CD206 (macrophage mannose receptor 1) and CD209 (dendritic cell-specific ICAM-3-grabbing non-integrin 1), with CD206 showing the most robust difference between M1 and M2 phenotypes.

The effect of ACdEV on Mo phenotype is highlighted by the significantly decreased expression of CD40, CD64 and CD86 (compared to M1). It is further evidenced by the increased expression of CD11b and CD206 (compared to M1). These results demonstrate the clear ability of ACdEV to change the phenotype of MO macrophages (M0+EV) towards a pro-resolution M2. Confirmation of this phenotypic change is provided through our PCA analysis where mean fluorescence intensity for each antigen demonstrates the greatest similarities between the M2 MΦ and ACdEV-treated M0 MØ, with CD206 being most robustly aligned (PCA biplot, FIG. 4B).

Overall, our results provide evidence that extracellular vesicles secreted from leukocytes during apoptosis carry a pro-resolving panel of lipid mediators and biosynthetic enzymes that can regulate the lipid metabolome profile in macrophages and expression of surface markers supporting the macrophage M2 pro-repair phenotype. These immunomodulatory properties of ACdEV highlight their importance, especially when released during defined stages of acute inflammation.

### In Vivo Data

### Methods

### In Vivo House Dust Mite Model

All animal procedures were carried out in strict accordance with the approved protocol and recommendations for proper use and care of laboratory animals (Animals (Scientific Procedures) Act 1986). All experiments on animals were conducted according to United Kingdom Home Office regulations (project license P75A73BEB). All animal handling was performed by qualified personnel. All studies were performed and reported according to the revised ARRIVE guidelines.

Thirty female C57BI/6 mice (6-8 weeks old) were purchased from Charles River and housed at the Aston University central animal facility under specific pathogen-free conditions with a 12-h light-dark cycle. The mice were provided with food and water ad libitum. Allergic airway disease was induced using a previously described protocol [PMID: 15528378]. In brief, mice (n=36 in three independently performed experiments) were anesthetized with isoflurane (Sigma-Aldrich) before the administration of house dust mite allergen (HDM) on three consecutive days. HDM extract (Citeq, The Netherlands) was suspended in sterile phosphate buffered saline (PBS) at a final concentration of 2.5 mg/ml. Ten μl of the solution was administered intranasally; control mice (n=36) received 10 μl of sterile PBS using the same protocol.

On day 3 of the experiment, mice were also administered human T cell-derived EVs (prepared as described above), either systemically via an intraperitoneal injection (i.p. ; n=48 in three independently performed experiments) or locally via the intranasal route (i.n. ; n=48). The experiment was terminated 18 hours after EV delivery (on day 4).

### Preparation of Single Cell Suspension From Mouse Lung

The lungs were removed and placed in an Eppendorf tube with 0.5 ml DMEM pen/strep (1%). Lungs were finely minced with scissors, and 0.5 ml DMEM pen/strep with collagenase/dispase (Sigma-Aldrich) added before incubating at 37° C. for 60 min. The reaction was terminated using 1 ml FBS with EDTA (5 mM), and samples were kept on ice thereafter. Digested tissue was mechanically dissociated by passing through a 70 μm cell strainer (Miltenyi) using a syringe plunger, then washed twice in RPMI buffer (RPMI, pen/strep, HEPES (25 mM), EDTA (5 mM) and FBS (10%); centrifuged at 1200 rpm, 4° C., 5 min) to create a single cell suspension.

### Staining of Lung Cells for Flow Cytometry

Cells were diluted to 10-50×10⁶ cells/ml in staining buffer (PBS, 10% FBS, EDTA (5 mM), and Fc receptors were blocked with anti-CD16/32 (1:100, Biolegend) for 10 min on ice. Cells were aliquoted into Eppendorf tubes, centrifuged (1200 rpm, 5 min, 4° C.), and resuspended in staining buffer containing antibodies pre-conjugated to fluorophores (see below) for 30 min on ice in the dark. Cells were washed twice to remove unbound antibody before re-suspending in 200 μl PBS prior to performing flow cytometry.

### Flow Cytometry

Single colour compensation controls using VersaComp antibody capture beads (Beckman Coulter) and Fluorescence Minus One (FMO) controls using cells were prepared at the time of staining following the same protocol. Stained samples were stored at 4° C., protected from light. All flow cytometry analyses were performed on a Cytoflex flow cytometer equipped with 405 nm (BV 421), 488 nm (530/30—FITC, 695/40—PerCp-Cy5.5), 561 nm (585/15—PE) and 640 nm (670/14—APC, APC/Cy7, AlexaFluor700) lasers and filters. The following markers were used to distinguish specific immune cell types: SiglecF—BV421, Ly6C—FITC, CD45—PerCP Cy5.5, CD64—PE, Ly6G—APC, CD11b—APC/Cy7, and CD11c—AlexaFluor700 (all from Biolegend). Data were analysed using FlowJo (BD Biosciences) software.

### Results

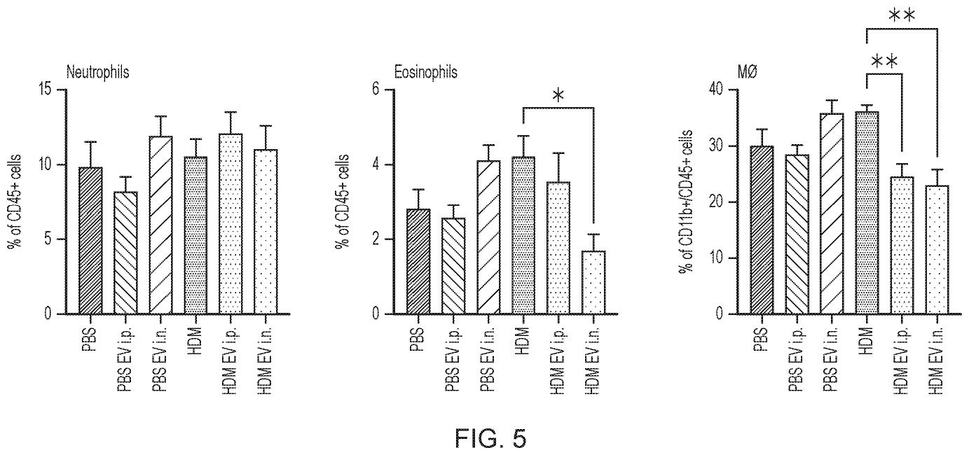

Following dissociation of the lung, multicolour staining of key immune cells within the lung preparation was undertaken and the presence of neutrophils, eosinophils, and inflammatory macrophages was assessed using the Beckman Coulter Cytoflex S. The results are shown in FIG. 6. Neutrophils were detected as CD11b+/Ly6G+ cells and expressed as a percentage of CD45+ cells. Eosinophils were detected as SiglecF+ cells and expressed as a percentage of CD45+ cells. Inflammatory macrophages were detected as Ly6C+ cells as a percentage of CD11b+/CD45+ cells.

The experiment was undertaken as two independent replicates providing 6 animals in each treatment group. Analysis of these data indicates that there is a significant decrease in inflammatory macrophages within the lung 18 hours after EV delivery. This occurred irrespective of whether delivery was local (intra-nasal) or systemic (intra-peritoneal). EV also reduced eosinophil numbers in the lung but this was only noted with local, intra-nasal delivery. The EV delivery showed no impact on neutrophil presence though this is perhaps expected given low neutrophil numbers at this time point in the inflammatory lung model.

These data suggest that EV have a beneficial effect in this in vivo model of lung inflammation.

### Discussion

Apoptotic cell clearance by phagocytes is an exquisitely efficient in vivo process, crucial for tissue homeostasis, and is known to support actively, anti-inflammatory responses [refs Fadok and later in vivo work]. Careful orchestration by dying cells enables successful intercellular communication with the innate immune system though remarkably little is known of the specific molecular detail of the communication events that drive phagocyte (macrophage) recruitment, nor the potential impact on macrophage phenotype that is known to change with removal of apoptotic cell corpses⁵⁸. It is established that shortly after the apoptosis onset, dying cells actively modify their plasma membranes to facilitate their phagocytic recognition and uptake^{24,59,60,61}. However, during apoptosis cells release rapidly significantly increased numbers of EV of various sizes^{62,63}, and while the large apoptotic bodies are removed rapidly by local cells (both professional and amateur phagocytes)¹, smaller ACdEV (e.g. exosomes and microvesicles/microparticles) may persist to travel longer distances and communicate apoptosis to attract more distant immune cells within and beyond the local tissue microenvironment. Previously, we demonstrated that smaller, apoptotic-bodies-depleted, ACdEV populations released form apoptotic B cells induce macrophage chemotaxis to the sits of leukocyte cell death, while ICAM-3 on their surface promotes macrophage chemoattraction and tethering¹⁷. This work provided some molecular mechanism to the process of macrophage recruitment to enrich the earlier studies showing a role for released membrane fragments¹⁵ and EV-associated CX3CL1¹⁶.

Here we further investigated ACdEV released from apoptotic lymphocyte cells, with an aim to define their composition more broadly while keeping a clear focus on ACdEV's ability to modulate immune responses and actively promote resolution of inflammation.

We demonstrate that induction of T cell apoptosis with anti-Fas/CHX promotes release of heterogeneous ACdEV populations, comprising a mixture of exosomes and microvesicles. Careful analysis of the ACdEV proteomic fingerprint revealed that ACdEV are enriched in the established EV major protein components, such as annexins, tetraspanins, Rabs, heat shock proteins, GTPases, 14-3-3 and HLA proteins²⁷. Importantly, ACdEV display ‘flags’ such as annexin A1 and calreticulin, including phosphatidylserine (PS) all of which are previously noted as ‘eat me’ signals carried on the surface of apoptotic cell corpses. Recognition, binding and uptake of apoptotic cells stimulates macrophage production of anti-inflammatory TGF-β1, and suppression of pro-inflammatory TNF-α, IL-1β, IL-8, IL-10, LtC₄ and thromboxane B₂, important routes by which ACdEV may modulate immune responses⁶⁴. Indeed, preferential enrichment of protein machinery responsible for ACdEV uptake via clathrin-dependent (CLH1) and clathrin-independent endocytosis (RhoA, Rac1), including a range of adhesion proteins (e.g. CD44, αL integrins, beta2 integrins, and IgSF members including ICAM-3) suggests that ACdEV are well-equipped to ensure their efficient interaction with and internalisation by recipient cells. However, ACdEV also display a range of proteins, such as CD48, ICAM-3, CD226 and SLAMF6 that allow them to interact with monocytes/macrophages, T cells and NK cells, while potentially eliciting an array of immune responses^{17,35,36,37}. Similarly, ACdEV carry AL5AP (also known as FLAP) and LKHA4, both involved in the synthesis of pro-inflammatory lipid mediators of inflammation—leukotrienes, and capable of immunomodulation even when present in exosomes⁴⁹.

The production of ACdEV by leukocytes during an acute inflammatory episode could act to propagate that pro-inflammatory state though our work suggests that ACdEV may actively oppose that potential and promote a resolution phase. Perhaps notably, we detect ACdEV-associated ABC transporters (MDR1, MRP1) which opens up the possibility that ACdEV promote lipid mediator (LM) signal transduction for longitudinal control of inflammation. ABC transporters, in particular MRP1 could serve as efflux pumps that actively release bioactive LM to the extracellular space, which would further activate LM-specific GPCRs on neighbouring immune cells to modulate immune responses⁶⁵. Although MRP1 has been associated with the release of leukotriene C4, it is plausible that it could represent a channel for the release of structurally similar LM with both pro-inflammatory and dual-acting properties. Such activity of EV highlights a key unanswered question in t-e field—do EV change following release and are they active, both immunologically and metabolically?

Our work now highlights that EV ligation by recipient immune cell receptors, to activate recipient cell signalling or uptake, may not be the only route by which ACdEV exert immunomodulatory properties. Whilst delivery of ACdEV-bound cargo is known to play an important role in determining the fate of the recipient immune cell, our work focused on the independent bioactivity carried and delivered by vesicles. Firstly, we addressed the bioactive nature of ACdEV by targeted lipid metabolomics LC-MRM analysis, to identify that ACdEV carry free PUFA, intermediate products of LM biosynthesis (e.g. hydroxyl-PUFA) and both pro-inflammatory and analogues of dual-acting SPM* (FIG. 2). The presence of pro-inflammatory mediators such as PGE₂, PGD₂, PGF_{2α} and LTB₄ is perhaps unexpected given the well-established claim that apoptotic cells are non-phlogistic and pro-resolving in nature. However, PGD₂/PGE₂ may also act as a pro-resolving molecular ‘switch’, shifting the immune responses from pro-inflammatory towards pro-resolving⁹, which adds complexity for the interpretation of an overall ACdEV LM signature. Thus, rather than seeking to assign individual LM contributions, we addressed ACdEV LM signature as an overall balance of bioactive LM and showed strong LM expression in favour of pro-resolving SPM* (FIG. 2F).

Secondly, and following identification of the presence of transporters for LM, we hypothesised that EV may contain all the necessary metabolic machinery for LM production52. This work identifies ACdEV, for the first time, as a lipoxygenase-loaded active metabolic compartment, rich in LM and capable of immune modulation. We reveal that ACdEV carry PLA2 and active LOX enzymes, with the highest specific activity of 15-LOX, and lower specific activities of 12-LOX and 5-LOX. Furthermore, we demonstrate that once ACdEV are internalised by LOX^{ko} recipient cells, ACdEV bioactivity is demonstrated in the rise in the recipient cell levels of LOX-specific lipid metabolites, importantly without stimulation in the presence of increased levels of enzyme substrates. Taking into count higher levels of SPM* and the highest activity of 15-LOX, it is reasonable to assume that ACdEV are of pro-resolving phenotype, and once internalised by an immune cell, capable of stimulating SPM biosynthesis and driving a pro-resolution phenotype. Indeed, we showed that ACdEV drive pro-resolving (M2) macrophage phenotype by abolishing the LTB4 levels, while stimulating macrophage biosynthesis of 15-LOX/15-LOX SPM (FIG. 4A) The phenotypic change towards M2 phenotype was further confirmed by surface immunophenotyping of macrophages treated with ACdEV (FIG. 4B).

Whilst transfer of LOX activity was shown here with an immune focus, it may well provide an opportunity for immune modulation in cells that do not express LOX to significant levels (e.g. non-professional phagocytes, neighbouring tissue cells). Although enzyme carriage within EV has been shown in recent studies^{49,50,51}, the specific relevance of biosynthetic capability to the immune modulating function of EV has not been formalised. Seminal work by Esser et al. reported presence of LKHA4 in macrophage-derived exosomes and showed that upon biostimulation with an excess of LTA₄, EV granulocytes showed increased levels of products of LTA₄ metabolism suggesting the activity of EV-bound LKHA₄⁴⁹. Recent work by Fafian-Labora et al. ⁵¹ further supports the hypothesis that the uptake of EV, rather than the surface receptor activation may mediate downstream effects due to the delivery of EV-bound cargo. Specifically, the group has shown that glutathione-S-transferase is active in senescent cell-derived EV and capable of ameliorating senescence. Nevertheless, the potential for ACdEV to exert their effects more widely must be considered. Future work should consider the kinetic changes that may occur in the ACdEV from the time of release from a donor cell to the, perhaps distant, interaction with a recipient cell. This work challenges the notion that ACdEV can be studied as a ‘snapshot’ and temporal studies of EV over time are essential to provide a more holistic view of the ACdEV composition that may change over time and it raises the possibility that ACdEV may modulate the function of many cells on their journey, rather than simply recipient cell.
1. Kerr J F, Wyllie A H, Currie A R. Apoptosis: a basic biological phenomenon with wide-ranging implications in tissue kinetics. Br J Cancer 26, 239-257 (1972).
2. Feig C, Peter M E. How apoptosis got the immune system in shape. Eur J Immunol 37 Suppl 1, S61-70 (2007).
3. Botto M, Walport M J. C1q, Autoimmunity and Apoptosis. Immunobiology 205, 395-406 (2002).
4. Brown J M, Attardi L D. The role of apoptosis in cancer development and treatment response. Nature Reviews Cancer 5, 231-237 (2005).
5. Savill J. Apoptosis in resolution of inflammation. J Leukoc Bio/61, 375-380 (1997).
6. Serhan C N, Chiang N, Van Dyke T E. Resolving inflammation: dual anti-inflammatory and pro-resolution lipid mediators. Nat Rev Immuno/8, 349-361 (2008).
7. Peters-Golden M, Canetti C, Mancuso P, Coffey M J. Leukotrienes: Underappreciated Mediators of Innate Immune Responses. The Journal of Immunology 174, 589-594 (2005).
8. Ricciotti E, FitzGerald G A. Prostaglandins and inflammation. Arteriosclerosis, thrombosis, and vascular biology 31, 986-1000 (2011).
9. Levy B D, Clish C B, Schmidt B, Gronert K, Serhan C N. Lipid mediator class switching during acute inflammation: signals in resolution. Nat Immuno/2, 612-619 (2001).
10. Serhan C N, Savill J. Resolution of inflammation: the beginning programs the end. Nat Immuno/6, 1191-1197 (2005).
11. Ravichandran K S. Find-me and eat-me signals in apoptotic cell clearance: progress and conundrums. J Exp Med 207, 1807-1817 (2010).
12. Tkach M, Théry C. Communication by Extracellular Vesicles: Where We Are and Where We Need to Go. Cell 164, 1226-1232 (2016).
13. Robbins P D, Morelli A E. Regulation of immune responses by extracellular vesicles. Nat Rev Immuno/14, 195-208 (2014).
14. Caruso S, Poon I K H. Apoptotic Cell-Derived Extracellular Vesicles: More Than Just Debris. Front Immuno/9, 1486-1486 (2018).
15. Segundo C, Medina F, Rodríguez C, Martínez-Palencia R, Leyva-Cobián F, Brieva J A. Surface molecule loss and bleb formation by human germinal center B cells undergoing apoptosis: role of apoptotic blebs in monocyte chemotaxis. Blood 94, 1012-1020 (1999).
16. Truman L A, et al. CX3CL1/fractalkine is released from apoptotic lymphocytes to stimulate macrophage chemotaxis. Blood 112, 5026-5036 (2008).
17. Torr E E, et al. Apoptotic cell-derived ICAM-3 promotes both macrophage chemoattraction to and tethering of apoptotic cells. Cell Death Differ 19, 671-679 (2012).
18. Tsai W H, et al. CX3CL1(+) Microparticles Mediate the Chemoattraction of Alveolar Macrophages toward Apoptotic Acute Promyelocytic Leukemic Cells. Cellular Physiology and Biochemistry 33, 594-604 (2014).
19. Bilyy R O, et al. Macrophages discriminate glycosylation patterns of apoptotic cell-derived microparticles. J Biol Chem 287, 496-503 (2012).
20. Devitt A, Pierce S, Oldreive C, Shingler W H, Gregory C D. CD14-dependent clearance of apoptotic cells by human macrophages: the role of phosphatidylserine. Cell Death & Differentiation 10, 371-382 (2003).
21. Théry C, et al. Minimal information for studies of extracellular vesicles 2018 (MISEV2018): a position statement of the International Society for Extracellular Vesicles and update of the MISEV2014 guidelines. Journal of Extracellular Vesicles 7, 1535750 (2018).
22. Fadok V A, Voelker D R, Campbell P A, Cohen J J, Bratton D L, Henson P M. Exposure of phosphatidylserine on the surface of apoptotic lymphocytes triggers specific recognition and removal by macrophages. The Journal of Immunology 148, 2207-2216 (1992).
23. Bratton D L, Fadok V A, Richter D A, Kailey J M, Guthrie L A, Henson P M. Appearance of phosphatidylserine on apoptotic cells requires calcium-mediated nonspecific flip-flop and is enhanced by loss of the aminophospholipid translocase. J Bio/Chem 272, 26159-26165 (1997).
24. Fadok V A, Voelker D R, Campbell P A, Cohen J J, Bratton D L, Henson P M. Exposure of phosphatidylserine on the surface of apoptotic lymphocytes triggers specific recognition and removal by macrophages. Journal of immunology (Baltimore, Md: 1950) 148, 2207-2216 (1992).
25. Chen H, et al. Extracellular Vesicles from Apoptotic Cells Promote TGFβ Production in Macrophages and Suppress Experimental Colitis. Scientific Reports 9, 5875 (2019).
26. Kalra H, et al. Vesiclepedia: a compendium for extracellular vesicles with continuous community annotation. PLOS Bio/10, e1001450 (2012).
27. Raposo G, Stoorvogel W. Extracellular vesicles: exosomes, microvesicles, and friends. J Cell Biol 200, 373-383 (2013).
28. Arur S, et al. Annexin I is an endogenous ligand that mediates apoptotic cell engulfment. Dev Cell 4, 587-598 (2003).
29. Gardai S J, et al. Cell-surface calreticulin initiates clearance of viable or apoptotic cells through trans-activation of LRP on the phagocyte. Cell 123, 321-334 (2005).
30. Oldenborg P A, Zheleznyak A, Fang Y F, Lagenaur C F, Gresham H D, Lindberg F P. Role of CD47 as a marker of self on red blood cells. Science (New York, NY) 288, 2051-2054 (2000).
31. Brown S, Heinisch I, Ross E, Shaw K, Buckley C D, Savill J. Apoptosis disables CD31-mediated cell detachment from phagocytes promoting binding and engulfment. Nature 418, 200-203 (2002).
32. Kaksonen M, Roux A. Mechanisms of clathrin-mediated endocytosis. Nature Reviews Molecular Cell Biology 19, 313-326 (2018).
33. Costa Verdera H, Gitz-Francois J J, Schiffelers R M, Vader P. Cellular uptake of extracellular vesicles is mediated by clathrin-independent endocytosis and macropinocytosis. Journal of Controlled Release 266, 100-108 (2017).
34. Bruno S, et al. Mesenchymal stem cell-derived microvesicles protect against acute tubular injury. J Am Soc Nephro/20, 1053-1067 (2009).
35. Shibuya K, et al. CD226 (DNAM-1) is involved in lymphocyte function-associated antigen 1 costimulatory signal for naive T cell differentiation and proliferation. J Exp Med 198, 1829-1839 (2003).
36. McNerney M E, Lee K M, Kumar V. 2B4(CD 244 ) is a non-MHC binding receptor with multiple functions on natural killer cells and CD8+ T cells. Molecular immunology 42, 489-494 (2005).
37. Wu N, et al. A hematopoietic cell-driven mechanism involving SLAMF6 receptor, SAP adaptors and SHP-1 phosphatase regulates NK cell education. Nature Immunology 17, 387-396 (2016).
38. Mathy N L, et al. Interleukin-16 stimulates the expression and production of pro-inflammatory cytokines by human monocytes. Immunology 100, 63-69 (2000).
39. Calandra T, Roger T. Macrophage migration inhibitory factor: a regulator of innate immunity. Nature Reviews Immunology 3, 791-800 (2003).
40. Lu J F, Luk F, Gong J, Jaiswal R, Grau G E, Bebawy M. Microparticles mediate MRP1 intercellular transfer and the re-templating of intrinsic resistance pathways. Pharmacological research 76, 77-83 (2013).
41. Bebawy M, et al. Membrane microparticles mediate transfer of P-glycoprotein to drug sensitive cancer cells. Leukemia 23, 1643-1649 (2009).
42. Fransen J H, et al. Mouse dendritic cells matured by ingestion of apoptotic blebs induce T cells to produce interleukin-17. Arthritis Rheum 60, 2304-2313 (2009).
43. Berda-Haddad Y, et al. Sterile inflammation of endothelial cell-derived apoptotic bodies is mediated by interleukin-1alpha. Proc Natl Acad Sci USA 108, 20684-20689 (2011).
44. Schiller M, et al. Induction of type I IFN is a physiological immune reaction to apoptotic cell-derived membrane microparticles. Journal of immunology (Baltimore, Md: 1950) 189, 1747-1756 (2012).
45. Eguchi A, et al. Microparticles release by adipocytes act as “find-me” signals to promote macrophage migration. PLoS One 10, e0123110 (2015).
46. Niessen A, et al. Apoptotic-cell-derived membrane microparticles and IFN-alpha induce an inflammatory immune response. J Cell Sci 128, 2443-2453 (2015).
47. Steinhilber D. Lipoxygenases: An Introduction. In: Lipoxygenases in Inflammation (ed Steinhilber D). Springer International Publishing (2016).
48. Kuhn H, Banthiya S, van Leyen K. Mammalian lipoxygenases and their biological relevance. Biochimica et biophysica acta 1851, 308-330 (2015).
49. Esser J, et al. Exosomes from human macrophages and dendritic cells contain enzymes for leukotriene biosynthesis and promote granulocyte migration. J Allergy Clin Immunol 126, 1032-1040, 1040 e1031-1034 (2010).
50. Iraci N, et al. Extracellular vesicles are independent metabolic units with asparaginase activity. Nat Chem Biol 13, 951-955 (2017).
51. Fafián-Labora J A, Rodríguez-Navarro J A, O'Loghlen A. Small Extracellular Vesicles Have GST Activity and Ameliorate Senescence-Related Tissue Damage. Cell Metabolism, (2020).
52. Devitt A, Griffiths H R, Milic I. Communicating with the dead: lipids, lipid mediators and extracellular vesicles. Biochem Soc Trans 46, 631-639 (2018).
53. Halder L D, et al. Immune modulation by complement receptor 3-dependent human monocyte TGF-beta1-transporting vesicles. Nat Commun 11, 2331 (2020).
54. Rådmark O, Werz O, Steinhilber D, Samuelsson B. 5-Lipoxygenase: regulation of expression and enzyme activity. Trends in Biochemical Sciences 32, 332-341 (2007).
55. Borgeat P, Hamberg M, Samuelsson B. Transformation of arachidonic acid and homo-gamma-linolenic acid by rabbit polymorphonuclear leukocytes. Monohydroxy acids from novel lipoxygenases. J Bio/Chem 251, 7816-7820 (1976).
56. Mosser D M, Edwards J P. Exploring the full spectrum of macrophage activation. Nat Rev Immuno/8, 958-969 (2008).
57. Dalli J, Serhan C N. Specific lipid mediator signatures of human phagocytes: microparticles stimulate macrophage efferocytosis and pro-resolving mediators. Blood 120, e60-e72 (2012).
58. Wickman G, Julian L, Olson M F. How apoptotic cells aid in the removal of their own cold dead bodies. Cell Death & Differentiation 19, 735-742 (2012).
59. Fadeel B. Plasma membrane alterations during apoptosis: role in corpse clearance. Antioxidants & redox signaling 6, 269-275 (2004).
60. Arur S, et al. Annexin I Is an Endogenous Ligand that Mediates Apoptotic Cell Engulfment. Developmental Cell 4, 587-598 (2003).
61. Païdassi H, et al. Investigations on the C1q-Calreticulin-Phosphatidylserine Interactions Yield New Insights into Apoptotic Cell Recognition. Journal of Molecular Biology 408, 277-290 (2011).
62. Théry C, et al. Proteomic Analysis of Dendritic Cell-Derived Exosomes: A Secreted Subcellular Compartment Distinct from Apoptotic Vesicles. The Journal of Immunology 166, 7309 (2001).
63. Tucher C, et al. Extracellular Vesicle Subtypes Released From Activated or Apoptotic T-Lymphocytes Carry a Specific and Stimulus-Dependent Protein Cargo. Front Immunol9, 534 (2018).
64. Fadok V A, Bratton D L, Konowal A, Freed P W, Westcott J Y, Henson P M. Macrophages that have ingested apoptotic cells in vitro inhibit proinflammatory cytokine production through autocrine/paracrine mechanisms involving TGF-beta, PGE2, and PAF. J Clin Invest 101, 890-898 (1998).
65. Robbiani D F, Finch R A, Jager D, Muller W A, Sartorelli A C, Randolph G J. The leukotriene C(4) transporter MRP1 regulates CCL19 (MIP-3beta, ELC)-dependent mobilization of dendritic cells to lymph nodes. Cell 103, 757-768 (2000).

1. A method of treating an inflammatory condition or disease comprising administering a pharmaceutically effective amount of an extracellular vesicle (EV),
wherein the EV comprises 15-lipoxygenase (15-LOX) and/or a nucleic acid encoding 15-LOX, and
optionally further comprising one or more of 12-lipoxygenase (12-LOX) and 5-lipoxygenase (5-LOX), and/or one or more nucleic acids encoding one or more of 12-LOX and 5-LOX.

2. An extracellular vesicle (EV) for use to treat an inflammatory disease or condition, the EV comprising 15-lipoxygenase (15-LOX), and/or a nucleic acid encoding 15-LOX, and
optionally further comprising one or more of 12-lipoxygenase (12-LOX) and 5-lipoxygenase (5-LOX), and/or one or more nucleic acids encoding one or more of 12-LOX and 5-LOX.

3. The method of claim 1, wherein the inflammatory disease is a chronic inflammatory disease.

4. The method of claim 1, wherein the inflammatory condition or disease is selected from a chronic wound, a cutaneous inflammatory disease, an autoimmune disease, nephritis, aging, lung disease, liver disease, or dementia.

5. The method of claim 4, wherein the inflammatory disease is selected from an internal wound, an external wound, a diabetic wound, or systemic lupus erythematosus (SLE).

6. The method of claim 1, claim, wherein the EV does not comprise 5-lipoxygenase (5-LOX) or a nucleic acid encoding 5-LOX.

7. The method of claim 1, wherein the EV is an isolated naturally occurring EV or a non-naturally occurring EV.

8. The method of claim 7, wherein the EV is an apoptotic cell-derived extracellular vesicle (ACdEV) or a viable cell derived EV most typically a mesenchymal stem cell derived EV.

9. The method of claim 1, further comprising one or more markers capable of targeting MØ macrophages, typically proinflammatory macrophages.

10. The method of claim 1, wherein the EV comprises one or more eat me markers, selected from Annexin A1, calrecticulin, or phosphatidylserine.

11. The method of claim 1, wherein the EV comprises one or more adhesion molecules, selected from an IgSF member (most typically ICAM-3), CD44, integrin alpha-1, or integrin beta2.

12. The method of claim 1, wherein the EV does not comprise one or more leukotrienes, most typically arachidonate 5-lipoxygenase-activating protein (AL5AP) or leukotriene A-4 hydrolase (LKHA4).

13. The method of claim 1, wherein the EV has a size range of at least 1 nm, or 10-1000 nm, more typically 70-700 nm.

14. The method of claim 1, wherein the EV has a mode size of 100-200 nm, more typically 90-130 nm.

15. The method of claim 1, wherein the EV is administered to a subject, or is capable of being administered to a subject, intravenously, intraperitonially, topically, intranasally, via oral inhalation, preferably via a nebuliser, or via an inhaler.

16. An isolated extracellular vesicle (EV), wherein the EV comprises 15-lipoxygenase (15-LOX) and/or a nucleic acid encoding 15-LOX, and optionally further comprising one or more of 12-lipoxygenase (12-LOX), 5 lipoxygenase (5-LOX), and/or one or more nucleic acids encoding one or more of 15-LOX, 12-LOX and 5-LOX.

17. The EV of claim 16, wherein the EV does not comprise 5-lipoxygenase (5-LOX) or a nucleic acid encoding 5-LOX.

18. The EV of claim 16, wherein the EV is an isolated naturally occurring EV or a non-naturally occurring EV.

19. The EV of claim 18, wherein the EV is an apoptotic cell-derived extracellular vesicle (ACdEV) or a mesenchymal stem cell EV.

20. The EV of claim 16, further comprising a pharmaceutically acceptable excipient.

21. The EV of claim 16, further comprising one or more markers capable or targeting proinflammatory macrophages.

22. The EV of claim 16, wherein the EV comprises one or more eat me markers, selected from Annexin A1, calrecticulin, or phosphatidylserine.

23. The EV of claim 16, wherein the EV comprises one or more adhesion molecules, selected from an IgSF member (most typically ICAM-3), CD44, integrin alpha-1, or integrin beta 2.

24. The EV of claim 16, wherein the EV does not comprise one or more leukotrienes, most typically arachidonate 5-lipoxygenase-activating protein (AL5AP, also known as FLAP) or leukotriene A-4 hydrolase (LKHA4).

25. The EV of claim 16, wherein the EV has a size range of at least 1 nm, typically 10-1000 nm, more typically 70-700 nm.

26. The EV of claim 16, wherein the EV has a mode size of 100-200 nm, more typically 90-130 nm.

27. The EV of claim 16, wherein the EV capable of being administered to a subject intraperitonially, topically, intranasally, via oral inhalation, preferably via a nebuliser, or via an inhaler.
